(19) United States
(12) Patent Application Publication
(10) Pub. No.: US 2023/0293559 A1
(43) Pub. Date: Sep. 21, 2023

(54) PHOSPHOLIPID BILAYERS CATALYTICALLY PROMOTE PROTEIN REFOLDING, INHIBIT AND REVERSE PROTEIN AGGREGATE FORMATION, AND METHODS OF TREATING NEURODEGENERATIVE DISEASES USING THE SAME

(71) Applicant: THE REGENTS OF THE UNIVERSITY OF COLORADO A BODY CORPORATE, Denver, CO (US)

(72) Inventors: Joel L. Kaar, Lafayette, CO (US); Daniel K. Schwartz, Boulder, CO (US); Andres Felipe Chaparro Sosa, Boulder, CO (US); Sabrina Matos De Oliveira Da Silva, Sao Carlos (BR); Victoria Reichelderfer, Boulder, CO (US)

(21) Appl. No.: 18/017,074

(22) PCT Filed: Aug. 13, 2021
(86) PCT No.: PCT/US21/46022

§ 371 Date: Jan. 19, 2023

Related U.S. Application Data
(60) Provisional application No. 63/065,121, filed on Aug. 13, 2020.

(51) Int. Cl. A61K 31/685; A61P 25/28

(52) U.S. Cl. CPC A61K 31/685; A61P 25/28

(57) ABSTRACT

Here, the present inventors describe novel methods and compositions to reduce protein misfolding, the formation of protein aggregates, as well as the degradation of previously formed protein aggregates, for example by separating fibrils back into protofilaments. Additional aspects of the invention include therapeutic uses of lipid bilayers to rescue misfolded proteins in Alzheimer's and other protein misfolding diseases.

### CROSS-REFERENCE TO RELATED APPLICATIONS

This International PCT Application claims the benefit of and priority to U.S. Provisional Application No. 63/065,121 filed Aug. 13, 2020. The entire specification and figure of the above-referenced application is hereby incorporated, in their entirety by reference.

### GOVERNMENT INTEREST

This invention was made with government support under grant number AG062979 awarded by the National Institutes of Health. The government has certain rights in the invention.

### TECHNICAL FIELD

The inventive technology is related to the field protein misfolding and neurodegenerative disease associated with protein misfolding, and in particular include systems, methods, and compositions for the use of homogenous and/or heterogenous (sometimes referred to as mixed) phospholipid bilayers to catalytically promote protein refolding, increase protein stability, prevent loss of protein secondary structure. Additional embodiments include systems, methods, and compositions for the use of phospholipid bilayers to catalytically inhibit and/or reverse the formation of protein aggregates. Additional embodiments include systems, methods, and compositions for the use of homogenous and/or heterogenous phospholipid bilayers to catalytically inhibit or reverse insoluble protein aggregates by promoting protein re-folding into soluble configurations. Finally, certain embodiments of the invention include methods of treating neurological and other disorders through the application of phospholipid bilayers to catalytically promote protein refolding, increase protein stability, prevent loss of protein secondary structure and the inhibition and/or reversal of protein aggregates associated with neurodegenerative and other diseases.

### BACKGROUND

Misfolding and aggregation of proteins are linked to diverse neurodegenerative diseases (e.g. Alzheimer's, Parkinson's, Creutzfeldt-Jakob). For example, Alzheimer's disease is characterized by the accumulation of neurofibrillary tangles (tau τ protein) and neuritic plaques (amyloid-β or Aβ) in the brain affecting especially the degeneration of neurons in the olfactory bulb and its connected brain structures. There is longstanding interest in the effects of biological interfaces, including cellular membranes, on the fibrillation of Aβ, in part due to the potential for these interfaces to serve as loci to enhance fibril nucleation and growth. While many approaches to reduce Aβ fibrillation have been aimed at slowing the formation of Aβ fibrils, the degradation of Aβ fibrils has remained a challenging task. However, there is also evidence that certain phospholipid bilayers may have neutral, or even inhibitory effects on protein fibrillation, including Aβ fibrillation. For example, vesicles with a variety of lipid and surfactant compositions (including anionic, cationic, and zwitterionic lipids) have been investigated, and while lipid bilayers are generally believed to influence the growth and stability of Aβ fibrils, the details and mechanisms of this connection remain only partially understood and constitute a potential direction for therapeutic treatment.

Prior work by Martins et al., showed that the treatment of mature Aβ fibrils with liposomes composed of DOPC, monosialotetrahexosylganglioside (GM1), and sphingomyelin as well as lipid extract from animal brain tissue led to the accumulation of soluble protofilaments. However, the resulting soluble protofilaments were not degraded further, and the structure of Aβ remained primarily β-sheet. Moreover, the soluble protofilaments proved to be more toxic than mature Aβ fibrils in mice, which was in agreement with the findings by Shea et al. Additionally, Friedman et al., reported that the interaction of Aβ fibrils with generic lipid vesicles led to the formation of oligomers in molecular dynamics simulations, although the oligomers were metastable and did not undergo further disaggregation nor was the re-folding of Aβ observed.

Compounding these problems, in lieu of treatments to reverse the progression of such protein misfolding associated neurodegenerative diseases, considerable effort has focused on detecting and developing therapeutic approaches to inhibit amyloid fiber formation during its initial stages. For example, there is substantial literature engineering antibodies, peptides, and small molecules to bind to soluble oligomers of amyloid-forming proteins to inhibit further oligomerization and further fiber formation. While such approaches have met with partial success, these approaches are stoichiometric, not catalytic, and there is a need for alternative strategies to inhibit even earlier stages of amyloid oligomerization by re-folding and thus rescuing misfolded proteins prior to aggregating as well as reverse the aggregation process altogether.

To address the problems, outlined above, the present inventors demonstrate the degradation of Aβ fibrils as well as the re-folding of Aβ by lipid bilayers. The present inventors demonstrate the use of homogenous and heterogenous lipid bilayers to increase protein stability, prevent loss of protein secondary structure, and inhibit protein aggregation (sometimes referred to as fibrillation herein). By demonstrating the therapeutic effect of lipid composition on protein misfolding, stability and fibrillation, and in particular A13 fibrillation and re-folding by homogenous and mixed DOPG/DOPC bilayers, the present invention may be widely and safely employed in preventing, and even reversing the progression of protein misfolding/aggregation associated diseases.

### SUMMARY OF THE INVENTION

One aspect of the invention may include novel systems, methods, and compositions configured to promote protein re-folding. In one preferred aspect, the present invention provides one or more lipid bilayer compositions that may catalytically promotes protein re-folding in an in vitro or in vivo system. In one preferred aspect, the lipid bilayer compositions may be homogenous, while in alternative embodiments the lipid bilayer compositions may include homogenous or heterogenous. In this preferred embodiment, the lipid bilayer compositions of the invention may include homogenous or heterogenous phospholipid bilayers of DOPC and/or DOPG.

Another aspect of the invention may include novel systems, methods, and compositions configured to promote protein stabilization. In one preferred aspect, the present invention provides one or more lipid bilayer compositions that promotes protein stabilization and/or loss of secondary structure in an in vitro or in vivo system. In one preferred aspect, the lipid bilayer compositions may be homogenous, while in alternative embodiments the lipid bilayer compositions may include homogenous or heterogenous.

Another aspect of the invention may include novel systems, methods, and compositions configured to inhibit protein fibrillization. In one preferred aspect, the present invention provides one or more lipid bilayer compositions that inhibit protein fibrillation in an in vitro or in vivo system. In one preferred aspect, the lipid bilayer compositions may be homogenous, while in alternative embodiments the lipid bilayer compositions may include homogenous or heterogenous.

Another aspect of the invention may include one or more lipid bilayer compositions to promote protein solubilization wherein the lipid bilayer catalytically promoting solubilization by re-folding of proteins associated with insoluble protein aggregates, such as inclusion bodies. Another aspect of the invention may include one or more lipid bilayer compositions that that inhibit or reverse the formation of protein aggregates, such as inclusion bodies by catalytically promoting solubilization by re-folding of proteins associated with insoluble protein aggregates, such as inclusion bodies.

Another aspect of the invention may include novel systems, methods and compositions configured to inhibit and/or reverse the formation of protein aggregates, and in particular protein aggregates, such as inclusion bodies, as well as increase protein stabilization. In one preferred embodiment, the present invention provides a composition configured to inhibit and/or reverse the formation of protein aggregates, wherein the composition comprises a lipid bilayer, and preferably a homogenous and/or homogenous or heterogenous phospholipid bilayer of DOPC and/or DOPG, that catalytically inhibits and/or reverses the formation of protein aggregates, such as fibrils and insoluble inclusion bodies, increases protein stabilization and loss of secondary structure and further inhibits protein aggregation.

In another preferred aspect, the present invention provides a composition for treating or preventing a disease or disorder associated with protein aggregates, wherein the composition comprises a lipid bilayer that catalytically inhibits the formation of said protein aggregates associated with a disease or disorder. In another preferred embodiment, the present invention provides a composition for treating or preventing a disease or disorder associated with protein aggregates, wherein the composition comprises a lipid bilayer that may catalytically reverse the formation of said protein aggregates associated with a disease or disorder.

In another preferred aspect, the present invention provides a composition for treating or preventing a disease or disorder associated with protein destabilization and/or aggregate formation, wherein the composition comprises a heterogenous or homogenous lipid bilayer that increase protein stabilization and inhibits the formation of protein aggregates associated with a disease or disorder. In another preferred embodiment, the present invention provides a composition for treating or preventing a disease or disorder associated with protein destabilization, loss of secondary structure, and the formation of protein aggregates, wherein the composition comprises a heterogenous or homogenous lipid bilayer that may catalytically reverse the formation of said protein aggregates associated with a disease or disorder.

One aspect of the invention may include novel systems, methods and compositions configured to promote protein re-folding. In one preferred embodiment, the present invention provides a composition for treating or preventing a disease or disorder associated with misfolded proteins, wherein the composition comprises a lipid bilayer, and preferably a single, or homogenous or homogenous or heterogenous phospholipid bilayer of DOPC/DOPG, that catalytically promotes protein re-folding associated with a disease or disorder.

In another aspect, the present invention provides a composition for treating or preventing a disease or disorder associated with protein aggregates, wherein the composition comprises a lipid bilayer, and preferably a homogenous or homogenous or heterogenous phospholipid bilayer of DOPC/DOPG that catalytically inhibits the formation of said protein aggregates associated with a disease or disorder, and/or increase protein stabilization and prevents loss of secondary structure that leads to fibrillation.

In another aspect, the present invention provides a composition for treating or preventing a disease or disorder associated with protein aggregates, wherein the composition comprises a lipid bilayer, and preferably a homogenous or heterogenous phospholipid bilayer of DOPC/DOPG, that may catalytically reverse the formation of said protein aggregates associated with a disease or disorder.

Another aspect of the invention may include novel systems, methods, and compositions for treating or preventing a disease or disorder associated with misfolded proteins, wherein the composition comprises a lipid bilayer, and preferably a homogenous or heterogenous phospholipid bilayer of DOPC/DOPG, that may catalytically promote protein re-folding associated with a disease or disorder.

Another aspect of the invention may include novel systems, methods, and compositions for treating or preventing a disease or disorder associated with protein instability and/or loss of secondary structure, wherein the composition comprises a lipid bilayer, and preferably a homogenous or heterogenous phospholipid bilayer of DOPC/DOPG, that may stabilize proteins associated with a disease or disorder.

In another preferred embodiment, the present invention provides a composition for treating or preventing a disease or disorder associated with protein aggregates, wherein the composition comprises a lipid bilayer, and preferably a homogenous or heterogenous phospholipid bilayer of DOPC/DOPG, that catalytically inhibits the formation of said protein aggregates associated with a disease or disorder.

In another preferred embodiment, the present invention provides a composition for treating or preventing a disease or disorder associated with protein aggregates, wherein the composition comprises a lipid bilayer, and preferably a homogenous or heterogenous phospholipid bilayer of DOPC/DOPG, that may catalytically reverse the formation of said protein aggregates associated with a disease or disorder.

In another aspect, the present invention provides a composition for treating or preventing a neurodegenerative disease or disorder, such as Alzheimer's disease associated with amyloid-β (Aβ) misfolding and/or the formation of Aβ fibrils, wherein the composition comprises a lipid bilayer, and preferably a heterogeneous lipid bilayer comprising DOPC or DOPG, that inhibits the formation of Aβ fibrils. In one preferred embodiment, a lipid bilayer of the invention, and preferably a heterogeneous lipid bilayer comprising DOPC or DOPG, may catalytically cause Aβ protein re-folding causing fragmentation of Aβ fibrils back into soluble protofilaments.

In another aspect, the present invention provides a composition for treating or preventing a neurodegenerative disease or disorder, such as Alzheimer's disease associated with amyloid-β (Aβ) misfolding and/or the formation of Aβ fibrils, wherein the composition comprises a lipid bilayer, and preferably a heterogeneous lipid bilayer comprising DOPC or DOPG, that reverses pre-formed Aβ fibrils. In one preferred embodiment, a lipid bilayer of the invention, and preferably a heterogeneous lipid bilayer comprising DOPC or DOPG, may catalytically decrease β-sheet content of Aβ fibrils in a subject.

Another aspect of the invention may include novel systems, methods, and compositions to inhibit the aggregation of insulin and/or α-synuclein, wherein the composition comprises a lipid bilayer, and preferably a homogenous or heterogenous phospholipid bilayer of DOPC/DOPG, that stabilize insulin and/or α-synuclein proteins in vitro or in vivo and prevent the loss of secondary protein structure.

Additional aspects of the inventive technology will become apparent from the specification, figures and claims below.

### BRIEF DESCRIPTION OF THE FIGURES

Aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. Inhibition of the formation of Aβ fibrils as a function of mixed DOPC/DOPG vesicle composition at 37° C. (A) ThT fluorescence of monomeric Aβ (11 μM) with and without (Ctl) vesicles as a function of time relative to fluorescence prior to incubation (i.e., relative fluorescence or F_{ThT}). (B) Difference in ellipticity of Aβ at 215 nm at time t and 0 h normalized to the ellipticity at 0 h (i.e., θ₂₁₅) as a function of incubation time. (C) Difference in relative F_{ThT} and θ₂₁₅ with and without vesicles after incubation for 24 h for each composition. Error bars represent the standard deviation from three independent measurements.

FIG. 2. Disruption of pre-formed Aβ fibrils as a function of mixed DOPC/DOPG vesicle composition at 37° C. (A) ThT fluorescence of monomeric Aβ (11 μM) with and without (Ctl) vesicles as a function of time relative to fluorescence prior to incubation (i.e., relative fluorescence or F_{ThT}). (B) Difference in ellipticity of Aβ at 215 nm at time t and 0 h normalized to the ellipticity at 0 h (i.e., 0215) as a function of incubation time. (C) Difference in relative F_{ThT} and 0215 with and without vesicles after incubation for 4 h for each composition. Error bars represent the standard deviation from three independent measurements.

FIG. 3. Rate constants for fibril degradation (k_{d}, black circles) and growth (k_{g}′, red triangles) as a function of DOPG content. Rate constants were determined by fitting the relative intensity data for F_{ThT} analysis of fibril degradation for each vesicle composition to a pseudo-first order reaction model. Error bars represent 68% confidence in the parameter fits.

FIG. 4. Negatively-stained transmission electron micrographs of pre-formed Aβ (1-42) fibers incubated with 50% DOPG at 37° C. (A) Before incubation with vesicles. (B) After incubation with vesicles for 1 h. (C) After incubation with vesicles for 24 h. Black as well as red arrows have been added to mark select fibrils and vesicles, respectively. The scale bar in the top images corresponds to a length of 100 nm. The images below each panel were collected at high magnification (scale bar=20 nm) to show the morphology of representative fibrils for each condition.

FIG. 5. Schematic representation of the tunable disruption of pre-formed Aβ fibrils as a function of mixed DOPC/DOPG vesicle composition.

FIG. 6. Complete CD spectra of monomeric Aβ incubated with mixed DOPG/DOPC vesicles for 0, 1, 1.75, 2.5, 4, and 24 h. Each spectrum represents the average from three separate scans in the far UV range.

FIG. 7. CD spectra of mature Aβ fibrils incubated with mixed DOPG/DOPC vesicles for 0, 1, 2, 4, and 24 h. Each spectrum represents the average from three separate scans in the far UV range.

FIG. 8. Fit of the relative intensity of ThT fluorescence (F_{ThT}) as a function of incubation time for Aβ fibrils in the presence of mixed DOPG/DOPC vesicles.

FIG. 9. Effect of lipid concentration on the disruption of pre-formed Aβ fibers for vesicles with 50% DOPG. (A) Relative intensity of ThT fluorescence (F_{ThT}) as a function of incubation time of Aβ fibrils (28 μM) with 1, 5, 11, and 20 mM vesicles. The lines represent best fits to the pseudo-first-order growth and degradation model used to understand the kinetics of the impact of lipid composition of fibrillation and fibril disruption. Error bars represent the standard deviation from three independent measurements. (B) Rate constants of fibril degradation (k_{d}, black circles) and growth (k_{g}′, red triangles) obtained from model fits of the concentration-dependent F_{ThT} data. Error bars represent 68% confidence in the parameter fits.

FIG. 10. Mean diameter of Aβ fibrils as a function of incubation time with 50% DOPG vesicles from analysis of TEM images. The mean diameter at time 0 h represents the diameter before incubation. Error bars represent the standard deviation of the average diameter of 11 to 22 individual fibers.

FIG. 11. Negatively-stained transmission electron micrographs of vesicles composed of 50% DOPG without Aβ fibrils. Images were obtained at different magnifications.

FIG. 12. Negatively-stained transmission electron micrographs of pre-formed Aβ fibers before, and after incubation with vesicles composed of 50% DOPG for 1 h, and 24 h. Scale bars represent 500 nm.

FIG. 13. Characterization of the inhibition of fibrillation of insulin via fluorescence using Thioflavin T (ThT). ThT fluorescence of insulin over time, showing the inhibition of amyloid fibrillization. Insulin is monomeric at time t=0 and stressed at 37° C. under agitation. 100% DOPC vesicles completely inhibit the fibrillization of insulin.

FIG. 14. Circular dichroism analysis of insulin structure in presence of lipid vesicles. CD signal of insulin over time, showing the retention in secondary structure. Insulin is monomeric at time t=0 and stressed at 37° C. under agitation. 100% DOPC vesicles completely retain the native secondary structure of insulin, indicated by the retention of CD signal at 208 nm over time.

FIG. 15. Raw circular dichroism spectra for each vesicle composition over 72 hours. For 100% DOPC vesicles, the characteristic peak for α-helix at 208 nm is completely retained over duration of analysis.

FIG. 16. Analysis of the inhibition of insulin aggregation by native polyacrylamide gel electrophoresis (PAGE). Native PAGE gels of insulin over time, showing the inhibition of amyloid fibrillization. Insulin is monomeric at time t=0 and stressed at 37° C. under agitation. The band for the native structure of insulin disappears as fibrillization increases. 100% DOPC vesicles completely inhibit the fibrillization of insulin, indicated by the retention of the band for the native structure of insulin.

FIG. 17. Characterization of the inhibition of fibrillation of α-synuclein via fluorescence using ThT. ThT fluorescence of α-synuclein over time, showing the inhibition of amyloid fibrillization. α-Synuclein is monomeric at time t=0 and stressed at 37° C. under agitation. All compositions of vesicles completely inhibit the fibrillization of α-synuclein.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

The present invention provides compositions and methods to treat or prevent a disease or disorder associated with misfolded proteins or protein aggregates. Specifically, the present invention is related to the discovery of the role of lipid bilayers in the catalytic promotion of protein refolding, as well as the inhibition and reversal of protein aggregates which play a role in the pathology of a variety of neurodegenerative disorders. For example, the accumulation and fibrillation of amyloid-β peptide (Aβ) within the brain tissue is deeply linked to the development of Alzheimer's disease. While many approaches to reduce Aβ fibrillation have been aimed at slowing the formation of Aβ fibrils, the degradation of Aβ fibrils has remained a challenging task. The present invention includes novel methods, systems, and compositions that in certain embodiment not only reduces the formation of fibrils, but more importantly degrades previously formed fibrils. The present inventors demonstrate that in one preferred embodiment when monomeric Aβ (1-42) was introduced to heterogeneous lipid vesicles composed of DOPC and DOPG, fibril formation was inhibited by as much as 76% in the presence of vesicles with a tunable lipid composition. In this embodiment, by tuning, or modifying the lipid composition of DOPC and DOPG in the lipid bilayer, the fibril content of pre-existing fibrils was furthermore decreased by as much as 74%. The present inventors further showed via transmission electron microcopy that after incubation with vesicles, pre-formed fibrils also decreased in diameter, indicating that the lipid bilayers of the invention catalyzed the degradation of fibrils by separating fibrils back into protofilaments. This embodiment demonstrates the potential therapeutic utility of heterogeneous lipid bilayers to rescue misfolded proteins in Alzheimer's and other protein misfolding diseases as describe below.

The present invention provides compositions and methods to inhibit misfolded proteins or protein aggregates. Specifically, the present invention is related to the discovery of the role of heterogenous, or mixed lipid bilayers, as well as homogenous lipid bilayers in the catalytic promotion of protein refolding, as well as the inhibition and reversal of protein fibrillation. For example, the aggregation of insulin is associated with injection localized amyloidosis, which impacts diabetic patients and can have severe clinical consequences. Additionally, the formation of amyloid fibers via α-synuclein is associated with Parkinson's disease and generates oligomeric species of α-synuclein that are believed to be highly toxic (similar to for amyloid-β in the case of Alzheimer's disease). As such, while insulin and α-synuclein are model proteins for studying the effect of the vesicles, they also have clinical significance.

The present invention includes novel methods, systems, and compositions that in certain embodiment increase protein stabilization and inhibits or reduces protein misfolding and fibrillation. In one preferred embodiment when monomeric insulin was introduced to heterogeneous lipid vesicles composed of DOPC and/or DOPG, the monomeric insulin showed increased retention of its secondary structure and further showed a reduction of amyloid fibrillization. In another preferred embodiment when monomeric insulin was introduced to homogeneous lipid vesicles composed of DOPC, which showed an increased retention in secondary structure of the monomeric insulin and a near complete inhibition of amyloid fiber formation. This embodiment demonstrates the potential therapeutic utility of heterogeneous and homogenous lipid bilayers to rescue clinically relevant misfolded proteins, such as insulin.

The present invention includes novel methods, systems, and compositions that in certain embodiment increase protein stabilization and inhibits or reduces protein misfolding and fibrillation in α-synuclein. In one preferred embodiment when monomeric α-synuclein was introduced to heterogeneous and homogenous lipid vesicles composed of DOPC and/or DOPG, the monomeric insulin showed increased retention of its secondary structure and further showed a nearly complete inhibition of amyloid fibrillization. This embodiment demonstrates the potential therapeutic utility of heterogeneous and homogenous lipid bilayers to rescue clinically relevant misfolded proteins, such as insulin and α-synuclein.

It will be appreciated by one of skill in the art, that the invention is not limited to treatment of a disease or disorder associated with protein misfolding or protein aggregates that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with protein misfolding or protein aggregates, in that a lipid bilayer composition, as discussed herein, can be administered to a subject prior to the onset of the disease or disorder, thereby preventing the disease or disorder.

Additionally, as disclosed elsewhere herein, one skilled in the art would understand that the present invention encompasses methods of treating, or preventing, a wide variety of diseases associated with protein misfolding or protein aggregates, where a lipid bilayer composition of the invention treats or prevents the disease. Various methods for assessing whether a disease is associated protein misfolding or protein aggregates are known in the art. Further, the invention encompasses treatment or prevention of such diseases discovered in the future.

One embodiment of the present invention includes novel, systems, methods, and compositions for the inhibition of amyloid fibril formation. In one preferred aspect of the invention, homogenous or mixed phospholipid vesicles may be used to catalytically inhibit amyloid fibril formation in a subject in need thereof. As detailed below, the inventive technology describes the therapeutic interactions between Aβ peptides/fibrils and unilamellar vesicles composed of mixtures of zwitterionic and anionic phospholipids, specifically DOPC and DOPG, respectively. Specifically, in this aspect, the fragment of Aβ consisting of residues 1-42, which is known to aggregate and form amyloid fibers.

In this aspect, the introduction of vesicles having tunable compositions of DOPC/DOPG mixtures significantly reduced the rate and extent of fibrillation. In another aspect, the introduction of vesicles having tunable compositions of DOPC/DOPG to fragment of Aβ consisting of residues 1-42, which is known to aggregate and form amyloid fibers, significantly reduced the rate and extent of fibrillation. In one preferred embodiment, vesicles with varying compositions of DOPC/DOPG also disrupted a significant fraction of pre-formed fibrils in as little as a few hours. As noted below, while disrupting the fibrils, the presence of the vesicles significantly reduced the β-sheet content of Aβ, suggesting the re-folding of Aβ to its native structure. These embodiments demonstrate the chaperone-like activity of homogenous or mixed DOPC/DOPG lipid bilayers towards Aβ and, moreover, demonstrate the potential therapeutic utility of DOPC/DOPG bilayers for the treatment of protein misfolding diseases generally.

For example, in one embodiment of the invention may include novel systems, methods and compositions configured to promote protein re-folding. In one preferred embodiment, the present invention provides a composition for treating or preventing a disease or disorder associated with misfolded proteins, wherein the composition comprises a lipid bilayer that may catalytically promotes protein re-folding associated with a disease or disorder. In another preferred embodiment, the present invention provides a composition for treating or preventing a disease or disorder associated with protein aggregates, wherein the composition comprises a lipid bilayer that catalytically inhibits the formation of said protein aggregates associated with a disease or disorder. In another preferred embodiment, the present invention provides a composition for treating or preventing a disease or disorder associated with protein aggregates, wherein the composition comprises a lipid bilayer that may catalytically reverse the formation of said protein aggregates associated with a disease or disorder.

In additional embodiments, the present invention provides a composition for treating or preventing a disease or disorder associated with protein misfolding and/or protein aggregates, wherein the composition comprises a lipid bilayer of zwitterionic and/or anionic phospholipids. In one preferred embodiment, the present invention provides a composition for treating or preventing a disease or disorder associated with protein misfolding and/or protein aggregates, wherein the composition comprises a lipid bilayer of 1,2-dioleoyl-sn-glycero-3-phosphocholine (DOPC) and/or 1,2-dioleoyl-sn-glycero-3-phospho-(1′-rac-glycerol) (DOPG). In one preferred embodiment, the lipid bilayer of invention may be a homologous lipid bilayer comparing DOPC or DOPG preferred while in alternative embodiments, the lipid bilayer of invention may include a heterogeneous lipid bilayer comparing comprising DOPC or DOPG. in this latter embodiment, the heterogeneous lipid bilayer may be tunable, such that the percentage of DOPC vs. DOPG present in the lipid bilayer may be adjusted.

In another embodiment, the present invention provides systems, methods, and compositions for delivering a lipid bilayer to a mis-folded protein, or a pre-formed protein aggregate, or a protein that may form a protein aggregate. In one preferred embodiment, a lipid bilayer of the invention, and preferably a heterogeneous lipid bilayer comprising DOPC or DOPG may form vesicle, which may preferably be a unilamellar vesicle. In one preferred embodiment, a lipid bilayer of the invention, and preferably a heterogeneous lipid bilayer comprising DOPC or DOPG may be disposed on a nano particle configured to secure said lipid bilayer.

In another embodiment, the present invention provides a composition for treating or preventing a neurodegenerative disease or disorder, such as Alzheimer's disease associated with amyloid-β (Aβ) misfolding and/or the formation of Aβ fibrils, wherein the composition comprises a lipid bilayer, and preferably a heterogeneous lipid bilayer comprising DOPC or DOPG, that inhibits the formation of Aβ fibrils. In one preferred embodiment, a lipid bilayer of the invention, and preferably a heterogeneous lipid bilayer comprising DOPC or DOPG, may catalytically cause Aβ protein re-folding causing fragmentation of Aβ fibrils back into soluble protofilaments. In another embodiment, a lipid bilayer of the invention may inhibit Aβ fibril formation by between 1% and 76% in a subject.

In another embodiment, the present invention provides a composition for treating or preventing a neurodegenerative disease or disorder, such as Alzheimer's disease associated with amyloid-β (Aβ) misfolding and/or the formation of Aβ fibrils, wherein the composition comprises a lipid bilayer, and preferably a heterogeneous lipid bilayer comprising DOPC or DOPG, that reverses pre-formed Aβ fibrils. In one preferred embodiment, a lipid bilayer of the invention, and preferably a heterogeneous lipid bilayer comprising DOPC or DOPG, may catalytically decrease β-sheet content of Aβ fibrils in a subject. In another embodiment, a lipid bilayer of the invention may reverse between 1% and 74% of pre-formed Aβ fibrils in a subject.

In another embodiment, the present invention provides a composition for treating or preventing a disease or disorder, associated with misfolding of tau, α-synuclein, and tumor suppressor protein p53, wherein the composition comprises a lipid bilayer, and preferably a heterogeneous lipid bilayer comprising DOPC or DOPG, that catalytically promotes protein re-folding of tau, α-synuclein, and tumor suppressor protein p53.

In another embodiment, the present invention provides a composition for treating or preventing a disease or disorder, associated with pre-formed protein aggregates of tau, α-synuclein, and tumor suppressor protein p53, wherein the composition comprises a lipid bilayer, and preferably a heterogeneous lipid bilayer comprising DOPC or DOPG, that catalytically reverses the formation of said pre-formed protein aggregates of tau, α-synuclein, and tumor suppressor protein p53 associated with a disease or disorder.

In another embodiment, the present invention provides a composition for treating or preventing a disease or disorder, associated with protein aggregates of tau, α-synuclein, and tumor suppressor protein p53, wherein the composition comprises a lipid bilayer, and preferably a heterogeneous lipid bilayer comprising DOPC or DOPG, that inhibits the formation of said protein aggregates of tau, α-synuclein, and tumor suppressor protein p53 associated with a disease or disorder.

Another embodiment of the invention includes methods for treating or preventing a disease or disorder associated with protein mis-folding in a subject in need thereof, the method comprising administering a therapeutically effective amount of a lipid bilayer of the invention to a subject, and preferably a heterogeneous lipid bilayer comprising DOPC or DOPG, wherein said lipid bilayer catalytically promotes protein re-folding associated with a disease or disorder.

Another embodiment of the invention includes a method for treating or preventing a disease or disorder associated protein aggregates in a subject in need thereof, the method comprising administering a therapeutically effective amount of a lipid bilayer of the invention to a subject, and preferably a heterogeneous lipid bilayer comprising DOPC or DOPG, wherein the lipid bilayer catalytically inhibits the formation of protein aggregates associated with a disease or disorder.

Another embodiment of the invention includes a method for treating or preventing a disease or disorder by the reversal of pre-formed protein aggregates in a subject in need thereof, the method comprising administering a therapeutically effective amount of a lipid bilayer to a subject, and preferably a heterogeneous lipid bilayer of the invention comprising DOPC or DOPG, wherein the catalytically reverses the formation of pre-formed protein aggregates associated with a disease or disorder.

Another embodiment of the invention includes methods for treating or preventing a neurodegenerative disease or disorder associated with protein mis-folding in a subject in need thereof, the method comprising administering a therapeutically effective amount of a lipid bilayer of the invention to a neural cell of the subject, and preferably a heterogeneous lipid bilayer comprising DOPC or DOPG, wherein said lipid bilayer catalytically promotes protein re-folding associated with a disease or disorder.

Another embodiment of the invention includes a method for treating or preventing a neurodegenerative disease or disorder associated protein aggregates in a subject in need thereof, the method comprising administering a therapeutically effective amount of a lipid bilayer of the invention to a neural cell of the subject, and preferably a heterogeneous lipid bilayer comprising DOPC or DOPG, wherein the lipid bilayer catalytically inhibits the formation of protein aggregates associated with a disease or disorder.

Another embodiment of the invention includes a method for treating or preventing a neurodegenerative disease or disorder by the reversal of pre-formed protein aggregates in a subject in need thereof, the method comprising administering a therapeutically effective amount of a lipid bilayer to a neural cell of the subject, and preferably a heterogeneous lipid bilayer of the invention comprising DOPC or DOPG, wherein the catalytically reverses the formation of pre-formed protein aggregates associated with a disease or disorder.

Another embodiment of the invention includes a method for treating or preventing a disease or disorder, the method comprising administering a therapeutically effective amount of a lipid bilayer, and preferably a heterogeneous lipid bilayer of the invention comprising DOPC or DOPG, to a subject in need thereof, wherein the disease or disorder is selected from the group consisting of: a polyQ disorder; a neurodegenerative disease or disorder selected from the group consisting of Spinocerebellar ataxia (SCA) Type 1 (SCA1), SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), a transmissible spongiform encephalopathy (prion disease), Creutzfeldt-Jakob disease (CJD), a tauopathy, and Frontotemporal lobar degeneration (FTLD); a disease or disorder selected from the group consisting of AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, injection localized amyloidosis, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis, and cancer associated with p53 mutant aggregates.

Another embodiment of the invention includes a method for treating or preventing a disease or disorder, the method comprising co-administering a therapeutically effective amount of a lipid bilayer, and preferably a heterogeneous lipid bilayer of the invention comprising DOPC or DOPG, to a subject in need thereof, with a therapeutic, pharmaceutical, biochemical, and biological agents or compounds for the treatment of a disease or disorder associated with protein mis-folding, and/or the formation protein aggregates,

Another embodiment of the invention includes a method for treating or preventing a disease or disorder, the method comprising co-administering a therapeutically effective amount of a lipid bilayer, and preferably a heterogeneous lipid bilayer of the invention comprising DOPC or DOPG, to a subject in need thereof, with a therapeutic, pharmaceutical, biochemical, and biological agents or compounds for the treatment of a disease or disorder treatable by the reversal of pre-formed protein aggregates, and/or the promotion of protein re-folding, and/or the inhibition of the formation of protein aggregates.

Protein misfolding has implications beyond those related to specific human disease or disorder. For example, protein misfolding and lack of solubility has been problematic in the generation of recombinant proteins. For example, one of the significant issues related to the expression of large amounts of recombinant proteins, for example in bacterial expression systems, is that many over-expressed proteins are unable to adopt a native, biologically-active conformation and thus become misfolded within the bacterial host cell. Generally misfolded proteins exhibit poor solubility and either accumulate in cells as insoluble aggregates (inclusion bodies) or are degraded by host cell proteases. Although most recombinant proteins that misfold are those that are non-native to the expression host cell, even native bacterial proteins can misfold and form insoluble aggregates during over-expression in bacterial recombinant protein expression systems. In another example, various therapeutic compositions rely in protein-based, and other biologic compounds to treat a host of disease and disorders. However, during the production, packaging and transfer of such therapeutics, there is a possibility that they may form undesirable aggregates.

Protein misfolding has implications related to the production of protein and other biologic therapeutic compounds. For example, protein therapeutics are popular and widely growing drug class, but the production, drug container, storage environment, transportation mechanism, and/or processing conditions in manufacturing can cause a variety of unintended, harmful protein aggregates to form in the drug product. Some protein aggregates can cause a decrease in efficacy of the expensive biopharmaceutical product and some aggregates can even cause adverse drug reactions such as unwanted immune responses, anaphylaxis, infusion reactions, complement activation, and even death. Hence it is crucial to monitor, detect, and more importantly prevent such protein aggregates in drug products and drug substances quickly.

To addresses these problems, the present invention includes compositions to promote protein solubilization and the inhibition and/or reversal of insoluble protein aggregates, such as inclusion bodies. In one embodiment, a lipid bilayer of the invention, may catalytically solubilize one or more insoluble misfolded proteins associated with insoluble protein aggregates, by promoting the re-folding of such proteins into solubilized forms. As generally described above, in this preferred embodiment, the lipid bilayer may be composed of zwitterionic and/or anionic phospholipids, such as DOPC and DOPG respectively, and may be introduced to an insoluble protein aggregate as a vesicle, such as unilamellar vesicle having a heterogeneous lipid bilayer of DOPC and DOPG. In alternative embodiments, a lipid bilayer of the invention may be composed of zwitterionic and/or anionic phospholipids, such as DOPC and DOPG respectively, and may be introduced to an insoluble protein aggregate through a nanoparticle, for example.

In still further embodiments, a lipid bilayer, and preferably a heterogeneous lipid bilayer of DOPC and DOPG, may be used in a protein expression system to prevent or reverse the formation of insoluble protein aggregates, such as inclusion bodies. For example, a lipid bilayer, and preferably a heterogeneous lipid bilayer of DOPC and DOPG, may be used to inhibit, or reverse the formation of inclusion bodies in prokaryotic cells, such as bacteria and yeast, and specifically bacteria and yeast engineered to produce high-level of wild-type, or recombinant proteins.

In other examples, a lipid bilayer, and preferably a heterogeneous lipid bilayer of DOPC and DOPG, may be used to inhibit, or reverse the formation of inclusion bodies in eukaryotic cells, such as in a human subject. In this embodiment, a lipid bilayer of the invention may be used for treating or preventing a disease or disorder associated with the formation of inclusion bodies in a subject in need thereof, the method comprising administering a therapeutically effective amount to said subject a lipid bilayer composition of the invention, and preferably a heterogeneous lipid bilayer of DOPC and DOPG, wherein said lipid bilayer promotes the solubilizing and re-folding of proteins associated with insoluble protein aggregates, and thereby inhibiting, or reversing the formation of insoluble protein aggregates.

In other examples, a lipid bilayer, and preferably a heterogeneous lipid bilayer of DOPC and DOPG, may be used to inhibit, or reverse the formation of inclusion bodies in in vitro assay or system. For example, in this embodiment, a lipid bilayer, and preferably a heterogeneous lipid bilayer of DOPC and DOPG, may be used to inhibit, or reverse the formation of inclusion bodies in an in vitro protein expression system, or in the preparation and storage of therapeutic compositions, such protein-based or other biologic pharmaceutical compounds that may be susceptible to the formation of insoluble protein aggregates. In this application, lipid bilayer composition of the invention, and preferably a heterogeneous lipid bilayer of DOPC and DOPG, is introduced to the in vitro assay or system and promotes the solubilizing and re-folding of proteins associated with insoluble protein aggregates, and thereby inhibiting, or reversing the formation of insoluble protein aggregates.

In other examples, a lipid bilayer, and preferably a heterogeneous lipid bilayer of DOPC and DOPG, may be used to inhibit, or reverse the formation of inclusion bodies in in vivo assay or system. For example, in this embodiment, a lipid bilayer, and preferably a heterogeneous lipid bilayer of DOPC and DOPG, may be used to inhibit, or reverse the formation of inclusion bodies in an in vivo protein expression system, such as a bioreactor or cell-free translation system. In this application, lipid bilayer composition of the invention, and preferably a heterogeneous lipid bilayer of DOPC and DOPG, is introduced to the in vivo assay or system and promotes the solubilizing and re-folding of proteins associated with insoluble protein aggregates, and thereby inhibiting, or reversing the formation of insoluble protein aggregates.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value. In addition, the term “between” includes all ranges within the stated number range provided. For example, throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

As used herein, “inhibits,” “inhibition” refers to the decrease in protein aggregation relative to the normal wild type level, or control level. Inhibition may result in a decrease in protein aggregation in response to the inhibition by a lipid bilayer of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “reverses,” “reverse” refers to the degradation of previously formed protein aggregates or fibrils relative to the wild type, or control level, for example a level associated with a stage of a disease condition. Reversal or protein aggregates or fibrils may result in a decrease in the number or composition of protein aggregates or fibrils in response to a lipid bilayer of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “reverses,” “reverse” refers also refers to a process whereby proteins undergo a structural change, for example where a protein undergoes a re-folding process to generate a protein in a new folded state that may have a different activity and/or structure In some embodiments, a reversed protein structure may include a protein folded such that it results in a disease or disorder associated with protein misfolding, while a reversed protein is a protein that has been re-folded to a wild-type or other structural configuration that is not associated with a disease or disorder, or a structural configuration that ameliorates the symptoms of a disease or disorder associated with protein misfolding. Reversal, with respect to protein re-folding, may result in an increase in the number or composition of refolded proteins in response to a lipid bilayer of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, a “lipid bilayer” refers to a lipid-containing membrane having two layers. A “phospholipid bilayer” refers to a lipid-containing membrane having two layers of phospholipids. Moreover, a lipid can be a biological lipid or a synthetic lipid. Non-limiting examples of lipids that can be used are gangliosides, sphingomyelins, cholesterol, dioleoyl-phosphatidylcholine (DOPC), dioleoyl-phosphatidyl serine (DOP S), dimyristoylphosphatidylcholine (DMPC), dimyristoylphosphatidylglycerol (DMPG), phosphatidylethanolamine (DSPE) and dioleoylphosphatidylethanolamine (DOPE). In another embodiment, the lipid is a membrane extract of biological cells

The term “vesicle” or “liposome” are terms of art to the skilled person. Typically, a vesicle is a small circular structure essentially consisting of aqueous fluid enclosed by a closed, spherical lipid bilayer. Crude membrane vesicles, however, are typically very divers and heterogeneous in size and content. They can either be specifically prepared or can form spontaneously upon cell/organelle disruption or lysis. Vesicles can differ in structure, size and/or composition. The structure of the vesicles can be unilamellar or multilamellar. Vesicles or liposomes include a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, the present invention also encompasses compositions that have different structures in solution than the normal vesicular structure. For example, the lipids may assume a micellar structure or merely exist as nonuniform aggregates of lipid molecules.

In a preferred embodiment, at least one lipid bilayer is administered to amyloid fibers when incorporated into a vesicle or nanoparticle. In another preferred embodiment, at least one lipid is administered to amyloid fibers when incorporated into a liposome. In order to produce liposomes of any kind, lipids need to be introduced into an aqueous environment. When dry lipid films are exposed to mechanical agitation in such an aqueous environment, large multilamellar vesicles are spontaneously formed. In order to produce smaller, uniformly sized and unilamellar vesicles (herein called liposomes in the examples), additional energy has to be dissipated into the system. The latter is often achieved by mechanical extrusion or by sonication. A general overview to manufacture liposomes is incorporated herein by reference (Reza M. Mozafari (2005) Cellular & Molecular Biology Letters 10, 711-719).

As used herein, “inclusion bodies” refer to nuclear or cytoplasmic aggregates of stainable substances, typically proteins. Proteins in inclusion bodies may be misfolded. “Inclusion body myocitis” refers to an age-related, inflammatory muscle disease, characterized by slowly progressive weakness and wasting of both distal and proximal muscles, most apparent in the muscles of the arms and legs. In sporadic inclusion body myositis, two processes, one autoimmune and the other degenerative, appear to occur in the muscle cells in parallel. The inflammation aspect is characterized by the cloning of T cells that appear to be driven by specific antigens to invade muscle fibers. The degenerative aspect is characterized by the appearance of vacuoles and deposits of abnormal proteins in muscle cells and filamentous inclusions.

As used herein, “protein aggregate” or “protein aggregates” are used to refer to proteins that are no longer dissolved, i.e, Aβ Protein aggregates can refer to agglomeration or oligomerization of two or more individual protein molecules but are not limited to such definitions. Protein aggregates used in the art may be soluble or insoluble, but unless specifically stated otherwise, protein aggregates are usually used for purposes of specific embodiments of the present invention considered insoluble. As used herein, “protein aggregate” or “protein aggregates” refers also to a “fibril” is a fibrillar aggregate of protein structures. As used herein, an “amyloid fibril” refers fibril containing a spherical structure comprising a Aβ peptides which appears to represent a series of spherical structures forming a curved structure.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “therapeutically effective amount” as used herein refers to that amount of a composition of the invention, and in particular a lipid bilayer of the invention, being administered which will relieve to some extent one or more of the symptoms of the disease or disorder being treated. In reference to the treatment of disease or disorder associated with misfolded proteins or protein aggregates, a therapeutically effective amount refers to that amount which has the effect of (1) reducing to some extent protein misfolding or increasing to some extent proper protein folding, (2) inhibiting to some extent protein misfolding, (3) inhibiting to some extent protein aggregation, and/or (4) reversing to some extent pre-formed protein aggregates or misfolded proteins, (5) relieving to some extent (or, preferably, eliminating) one or more signs or symptoms associated with the a disease or disorder associated with misfolded proteins or protein aggregates. For example, in some cases the lipid bilayer compositions of the present disclosure can be employed for the treatment of Alzheimer's disease, Alzheimer's disease (AD), Parkinson's disease, Huntington's disease, amyotrophic, lateral sclerosis (ALS), Lewy body dementia (LBD), or Down's syndrome. In some cases, the compounds of the present disclosure can be employed for the detection, diagnosis, treatment, and monitoring of Alzheimer's disease. Or the compounds of the present disclosure can be employed for the detection, diagnosis, treatment, and monitoring of Creutzfeldt-Jakob disease (CJD).

As used herein, “subject” refers to a human or animal subject. In certain preferred embodiments, the subject is a human.

A “composition” “compound” or “pharmaceutical composition” encompasses a combination of an active agent, such as a lipid bilayer as generally described herein, or diluents, binder, stabilizer, buffer, salt, lipophilic solvent, preservative, adjuvant or the like, or a mixture of two or more of these substances. Carriers are preferably pharmaceutically acceptable.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “co-administering” or co-administer” refers to the administration of a lipid bilayer of the invention with a therapeutic, pharmaceutical, biochemical, and biological agents or compounds for the treatment of a disease or disorder treatable by the protein re-folding, inhibition of protein aggregate formation, and/or reversal of pre-formed protein aggregates. The therapeutic, pharmaceutical, biochemical, and biological agents or compounds co-administered along with the above-described lipid bilayer of the invention and routes of delivery of this invention for the treatment of neurodegenerative and other diseases specific to the disease are many and diverse in nature. They may be selected from the group consisting of: The chemotherapeutics, insulin, IGF-1, levodopa (5-10% crosses BBB) combined with a dopa decarboxylase inhibitor or COMT inhibitor, dopamine agonists and MAO-B inhibitors (selegiline and rasagiline)), Dopamine agonists (include bromocriptine, pergolide, pramipexole, ropinirole, piribedil, cabergoline, apomorphine and lisuride), non-steroidal anti-inflammatory drugs, acetyl cholinesterase inhibitors (such as tacrine, donepezil and the longer-acting rivastigmine; antibiotics), 2,4-dinitrophenol, glutamate receptor antagonist, glutathione, NMDA-receptor blocker such as ketamine, R amyloid inhibitor besides bexarotene, Alzheimer's vaccine, non-steroidal anti-inflammatory drug including COX-2 inhibitor, deferoxamine, hormones such as progesterone, enzymes, erythropoietin, Intranasal fibroblast growth factor, epidermal growth factor, microglial activation modulator, cholinesterase inhibitor, stimulant of nerve regeneration, nerve growth factor, non-steroidal anti-inflammatory drugs, interferon-β (IFN-β), antioxidants, Zinc and magnesium L. threonate with hormone, vitamin B12, A, E, D3, and B complexes, inhibitor of protein tyrosine phosphatase and similar therapeutic agents.

Administration of a composition of the invention, and preferably administration of a lipid bilayer, and even more preferably a homogenous or heterogenous phospholipid bilayer of DOPC/DOPG, may be administered by any method that enables delivery of the lipid bilayer to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound, for heterogenous phospholipid bilayer of DOPC/DOPG, calculated to produce the desired therapeutic effect in association with the required pharmaceutical carrier. The specification for the dosage unit forms are dictated by and directly dependent on (a) the unique characteristics of the composition and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each composition to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the composition are well-known in the relevant art and would be understood to be encompassed by the skilled artisan once provided the teachings disclosed herein.

The amount of a lipid bilayer, and preferably a heterogenous phospholipid bilayer of DOPC/DOPG of the invention, administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day. In one preferred embodiment, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day. In some cases, the aforesaid dosage examples may describe a dosage range for a combination of a lipid bilayer, and preferably a heterogenous phospholipid bilayer of DOPC/DOPG, and another therapeutic composition.

As used herein, a lipid bilayer may be a “pharmaceutically acceptable carrier” which refers to a carrier or diluent that does not cause significant irritation to an organism and does not abrogate the biological activity and properties of the administered lipid bilayer, and preferably a heterogenous phospholipid bilayer of DOPC/DOPG of the invention. A pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Inhibition of the Formation of Aβ Fibrils as a Function of Mixed DOPC/DOPG Vesicle Composition

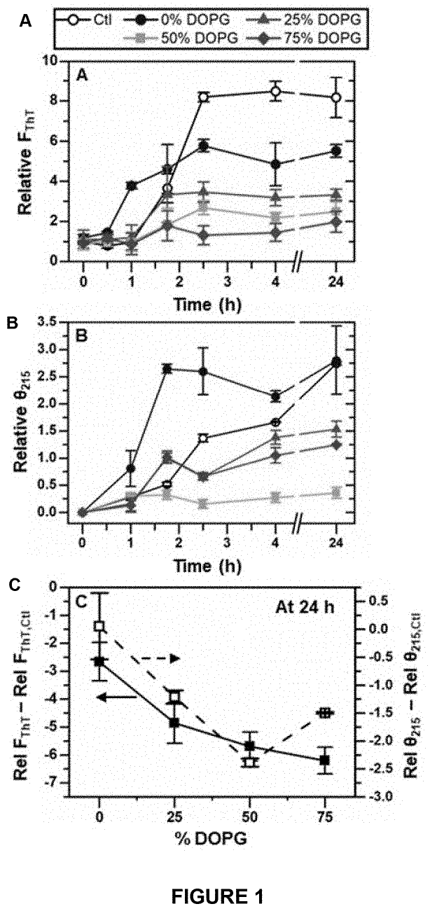

Inhibition of fibrillation of monomeric Aβ by mixed DOPC/DOPG vesicles with 0, 25, 50 and 75% DOPG content was initially determined using thioflavin T (ThT) and circular dichroism. Both time-dependent ThT assays and CD showed that formulated mixed lipid vesicles dramatically inhibited the formation of amyloid fibrils at 37° C. In the case of ThT assays, an initial induction period was observed followed by an increase in ThT fluorescence relative to the fluorescence prior to incubation (i.e., relative F_{ThT}) over a period of several hours (FIG. 1A). After 2-3 hours, the relative F_{ThT} saturated at a maximum value that depended on the vesicle composition. While even the least effective (100% DOPC) vesicle composition reduced fibril formation by 32%, the vesicles incorporating 50% or 75% DOPG were much more effective, reducing fibril formation by 70% and 76%, respectively.

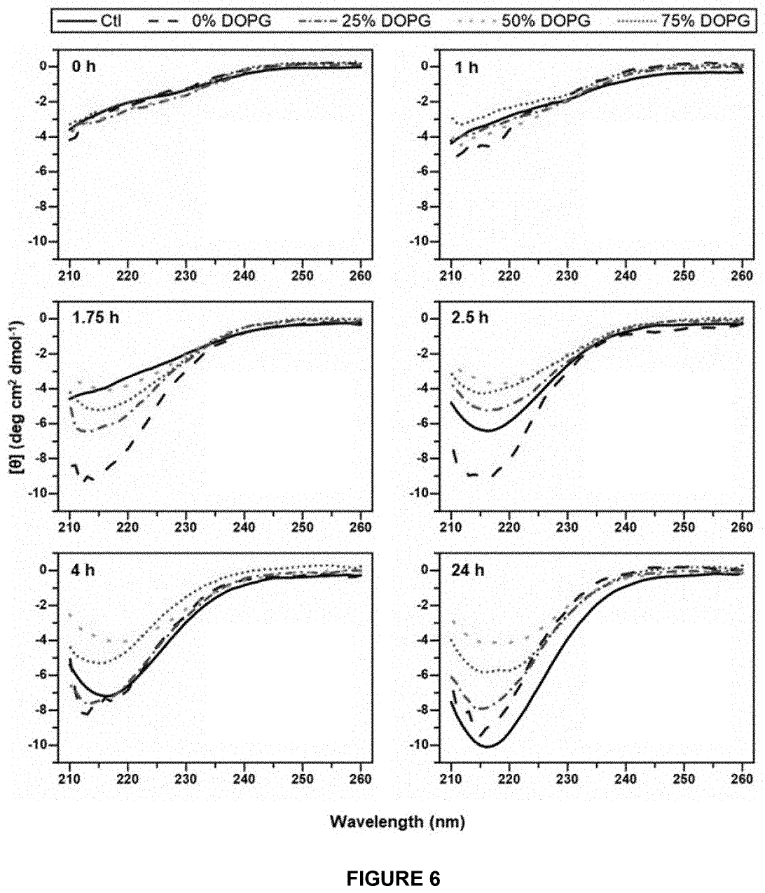

Characterization of the fibrillation process via CD provided qualitatively consistent findings, as shown in FIG. 1B, using the difference in ellipticity of Aβ at 215 nm at time t and 0 h normalized to the ellipticity at 0 h (i.e., relative θ₂₁₅) as a proxy for β-sheet formation (full spectra are shown in FIG. 6). While pure DOPC did not inhibit the β-sheet content of Aβ significantly, the formation of β-sheet structure decreased dramatically in the presence of vesicles with 50% DOPG (and to a lesser extent 25% and 75% DOPG). The effect of lipid bilayer composition is readily visualized in FIG. 1C, which shows that the difference in F_{ThT} and θ₂₁₅ from the control (without vesicles) after incubation for 24 h was the greatest for 50% and 75% DOPG vesicles.

### Example 2: Disruption of Pre-Formed Aβ Fibrils as a Function of Mixed DOPC/DOPG Vesicle Composition

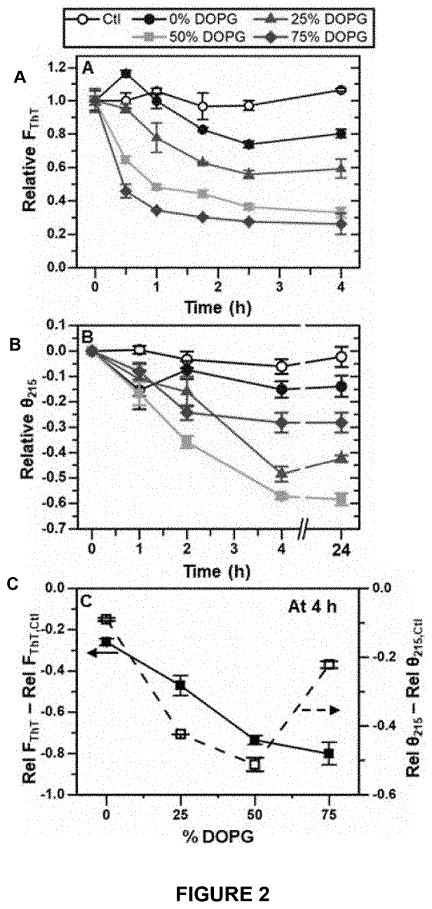

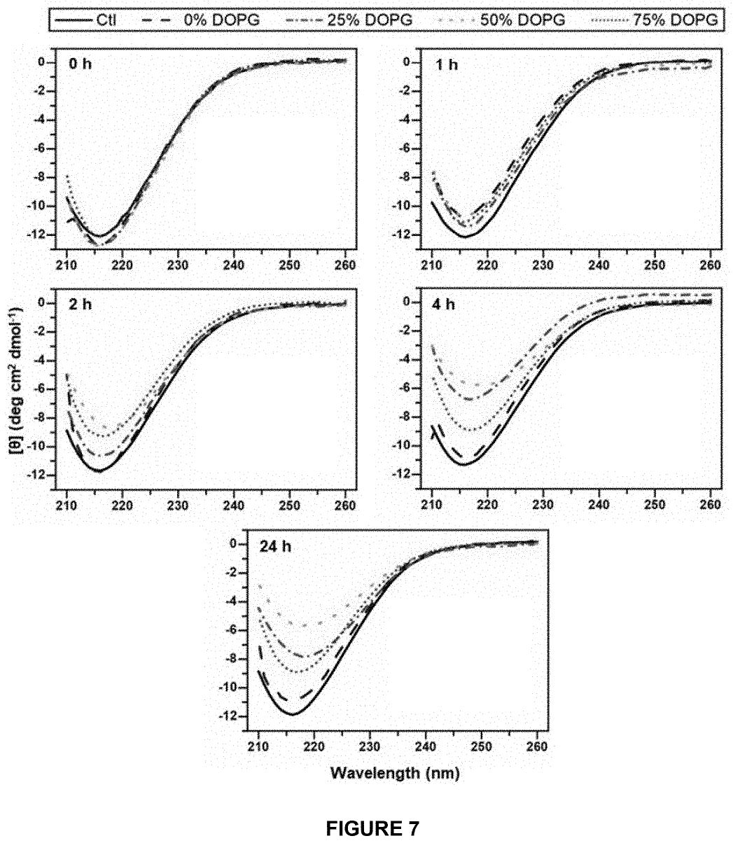

Having shown the ability of mixed DOPC/DOPG vesicles to prevent fibrillation, the present inventors sought to determine whether such vesicles also degraded pre-formed Aβ fibrils at 37° C. In the absence of mixed DOPC/DOPG vesicles, the relative F_{ThT} remained constant with time, consistent with the expected retention of stable fibrils (FIG. 2A). However, in the presence of vesicles, F_{ThT} decreased systematically with time at a rate that was sensitive to the phospholipid composition. While zwitterionic DOPC vesicles reduced F_{ThT} by only 20%, vesicles containing 50% and 75% DOPG reduced F_{ThT} by 67% and 74%, respectively, reflecting a substantial degradation of amyloid fibrils. Furthermore, analysis of the degradation of the fibrils by mixed DOPC/DOPG vesicles via CD showed that the β-sheet content of Aβ was significantly reduced (FIG. 2B). This was particularly evident in the case of vesicles with 50% DOPG for which the degradation of pre-formed Aβ fibrils was the greatest over time. For all mixed vesicles, the degradation of Aβ fibrils occurred predominately over the initial 4 h upon addition of the vesicles (full spectra are shown in FIG. 7). FIG. 2C shows the effectiveness of vesicles towards fibril disruption as a function of DOPG content. Overall, there was strong agreement between the ability of a particular vesicle composition to inhibit the formation of amyloid fibrils and its ability to degrade pre-formed fibrils.

Although there was general agreement between the ThT and CD measurements, the results differed significantly for the case of vesicles with 75% DOPG. A similar difference was also observed for the inhibition of fibril formation by vesicles with 75% DOPG (FIG. 1C). Since ThT and CD measure different, but complementary, structural features, this suggests that the disruption of Aβ fibrils may not always correlate perfectly with the secondary structure of Aβ (i.e., despite the disruption of fibrils, Aβ may remain largely β-sheet in structure). Likewise, it is plausible that some vesicles compositions may prevent fibrillation, but not Aβ misfolding.

Our results suggest that DOPG content had a significant impact on the extent of both fibril formation and degradation. The present inventors previously demonstrated that the stability and activity of enzymes tethered to mixed lipid bilayers was directly related to lipid composition. Furthermore, using single-molecule methods, the present inventors found that that the enhancement in enzyme stability and activity upon varying lipid composition was due to a chaperone-like effect of the bilayer, whereby the bilayer actively mediated the re-folding of denatured enzyme molecules at the bilayer-solution interface. In a similar manner, tuning the composition of mixed DOPG/DOPC vesicles may have a similar effect on the stabilization of Aβ. Specifically, by acting as a molecular chaperone, the mixed DOPG/DOPC vesicles may degrade Aβ fibrils and, moreover, catalyze Aβ re-folding. The dependence of this chaperone-like activity on vesicle composition may be the result of balancing the strength of the interaction of misfolded Aβ with the bilayer surface. If these interactions are too strong, the misfolded state may be stabilized, thereby inhibited re-folding, whereas, if too weak, the interactions may not be sufficient to overcome the energy barrier associated with Aβ re-folding.

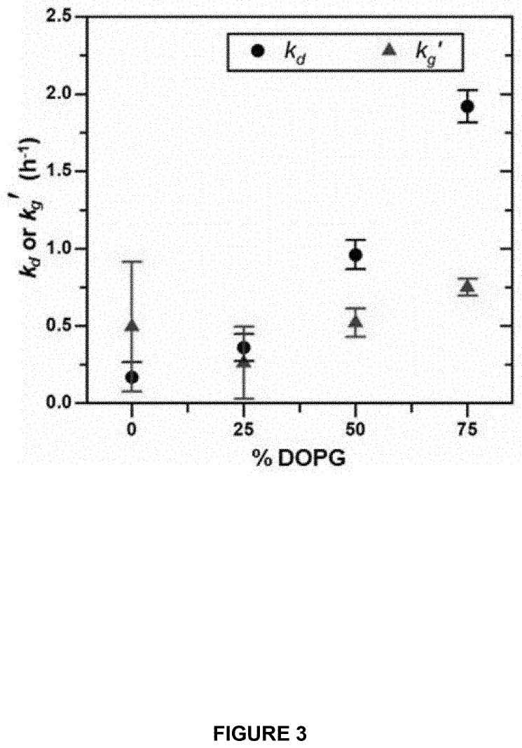

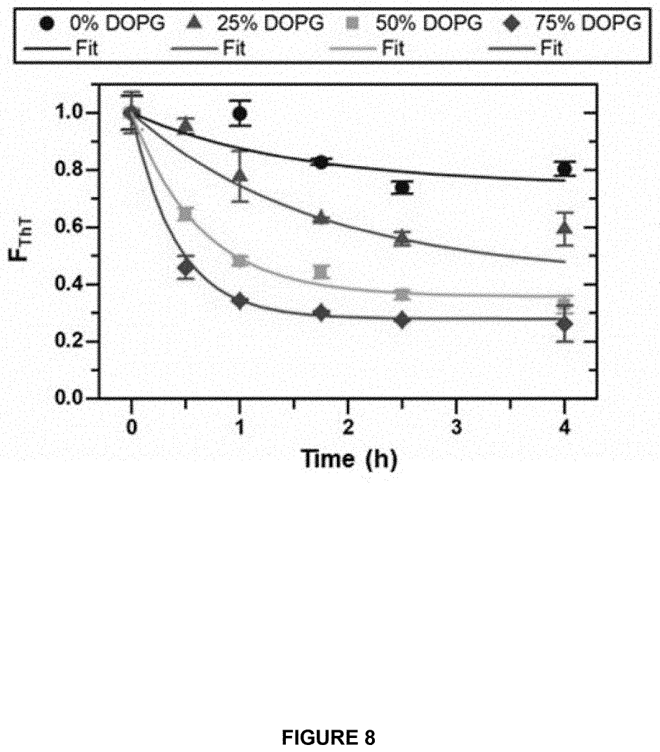

To provide a more mechanistic view of the chaperone-like activity of mixed DOPC/DOPG vesicles towards Aβ fibrils, the F_{ThT} data from FIG. 2A was fit to a pseudo-first-order growth and degradation model. In this model, fibril degradation is first-order in the concentration of Aβ in fibrils (with rate constant k_{d}) while fibril growth is pseudo-first-order in the concentration of monomeric Aβ (with rate constant k_{g}′); i.e. the model assumes that k_{g}′ is limited by the amount of monomeric Aβ in solution. As shown in FIG. 8, this model, although simple, accurately captures the important behavior appropriately, and agrees well with the experimental data in FIG. 2A. Interestingly, the results of the fitting analysis show that while k_{g}′ remained relatively constant across all vesicle compositions, k_{d} increased with higher DOPG content (FIG. 3). This observation suggests that increasing the DOPG content of the vesicles accelerated the degradation of fibrils while the rate of growth of fibrils was largely unchanged. Notably, although k_{g}′ remained relatively constant as a function of vesicle composition, the general trend for k_{g}′ followed that for k_{d} as the vesicle composition varied. The apparent increase in k_{g}′ with k_{d} as a function of vesicle composition is consistent with the mechanistic view of the bilayer as a catalyst with chaperone-like activity (where the catalyst accelerates both the forward and reverse reactions).

### Example 3: Effect of Lipid Concentration on the Disruption of Pre-Formed Aβ Fibers

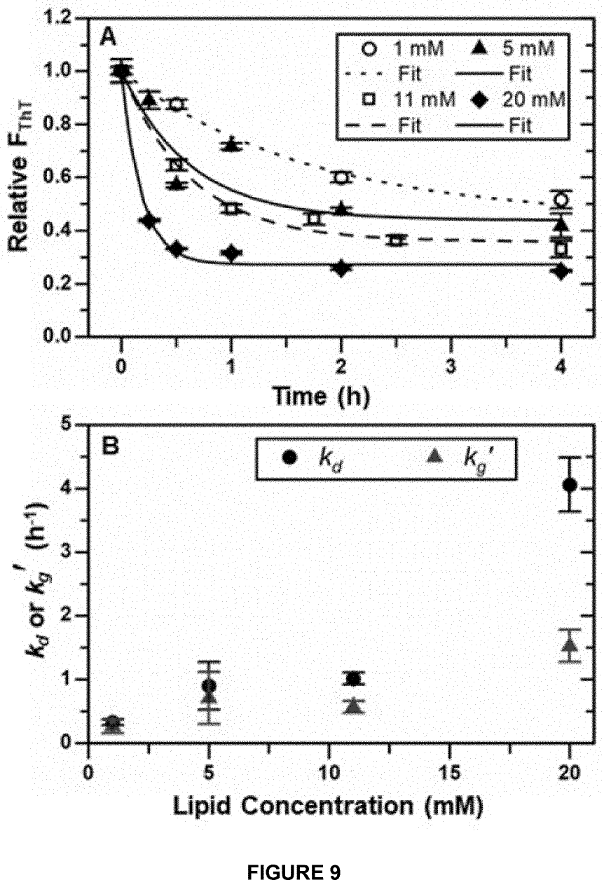

To further investigate the effect of mixed DOPC/DOPG vesicles on the degradation of pre-formed Aβ fibrils, the effect of lipid concentration on fibril degradation kinetics was quantified. For these studies, we employed vesicles with 50% DOPG since this was the optimal composition for both degrading pre-formed fibrils and inhibiting fibril formation. As expected, the rate of fibril disruption increased markedly as the concentration of lipids was increased from 1 mM to 20 mM (FIG. 9A). For example, whereas relative F_{ThT} decreased to 0.87 after 0.5 h with 1 mM lipids, incubation with 22 mM lipids led to a decrease in relative F_{ThT} to 0.33 in the same time. The effect of lipid concentration on the degradation of pre-formed fibrils was further shown by determining k_{d} as a function of lipid concentration from the concentration-dependent F_{ThT} data. As shown in FIG. 9B, k_{d} increased monotonically as a function of lipid concentration with a 12-fold difference between k_{d} at 20 mM (4.1 h⁻¹) and at 1 mM (0.33 h⁻¹). Interestingly, k_{g}′ also increased over this range of lipid concentration, although the magnitude of the increase in k_{g}′ (6-fold between 20 mM and 1 mM) was considerably less than that for k_{d}. As noted above, the increase in k_{g}′ with k_{d} as a function of lipid concentration is consistent with the presumed role of the vesicles as a catalyst.

### Example 4: Morphological Characteristics of Aβ Fibrils Before and After Treatment with DOPC/DOPG Vesicles

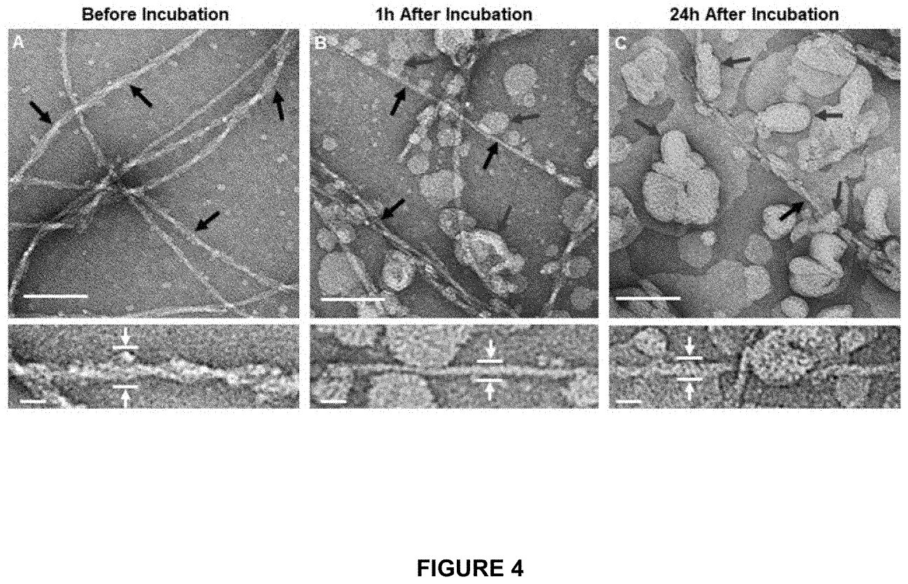

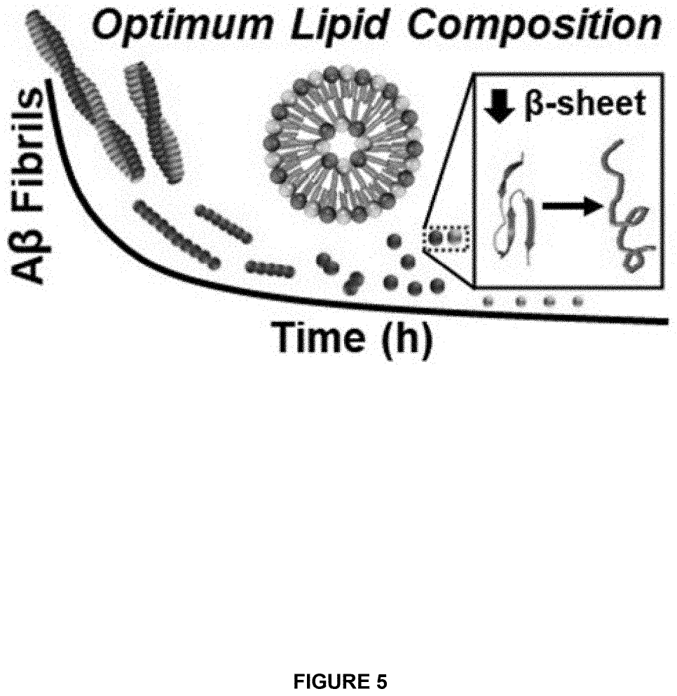

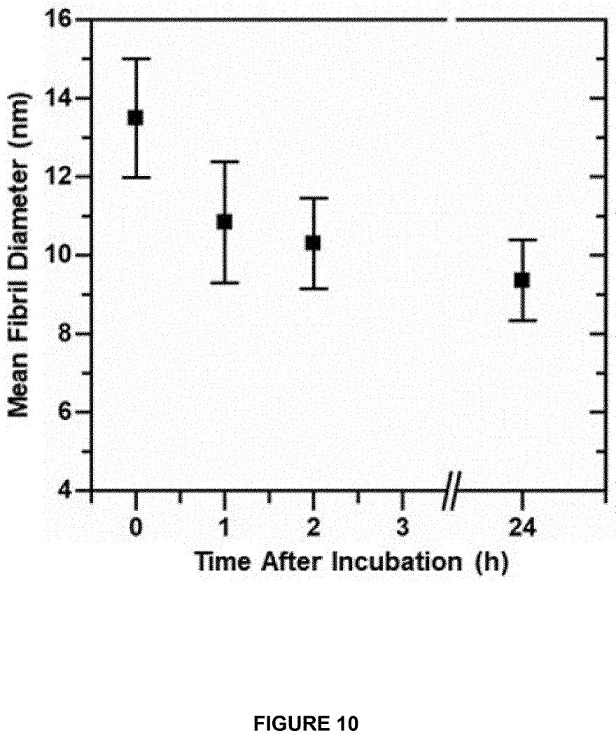

As further evidence of the ability of mixed DOPC/DOPG vesicles to degrade pre-formed Aβ fibrils, the morphology of Aβ fibrils before and after treatment with vesicles at 37° C. was characterized by transmission electron microscopy (TEM). Prior to incubation with vesicles, Aβ fibrils, which were comprised of multiple protofilaments intertwined with one another, exhibited a helical ribbon structure as reported previously (FIG. 4A). Given the oscillatory nature of the helical structure of fibrils, the width of the fibers varied between 24 nm at the widest and 7 nm at the narrowest locations, respectively. However, incubation of the fibrils with mixed DOPG/DOPC vesicles resulted in the apparent loss of the ribbon morphology, indicating disruption of the fibrils. The loss of this morphology can be clearly seen in FIGS. 4B and C, which show representative images of fibrils incubated with 50% DOPG vesicles for 1 h and 24 h, respectively. Comparison of fibril diameter upon incubation with 50% DOPG vesicles found that the mean fibril diameter decreased from 14±2 nm (n=18 fibrils) before incubation to 9±1 nm (n=11 fibrils) after 24 h (FIG. 10).

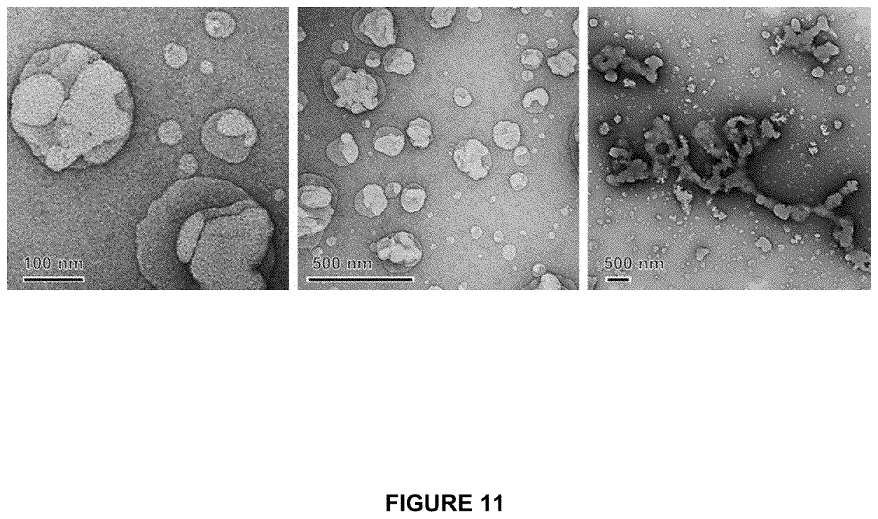

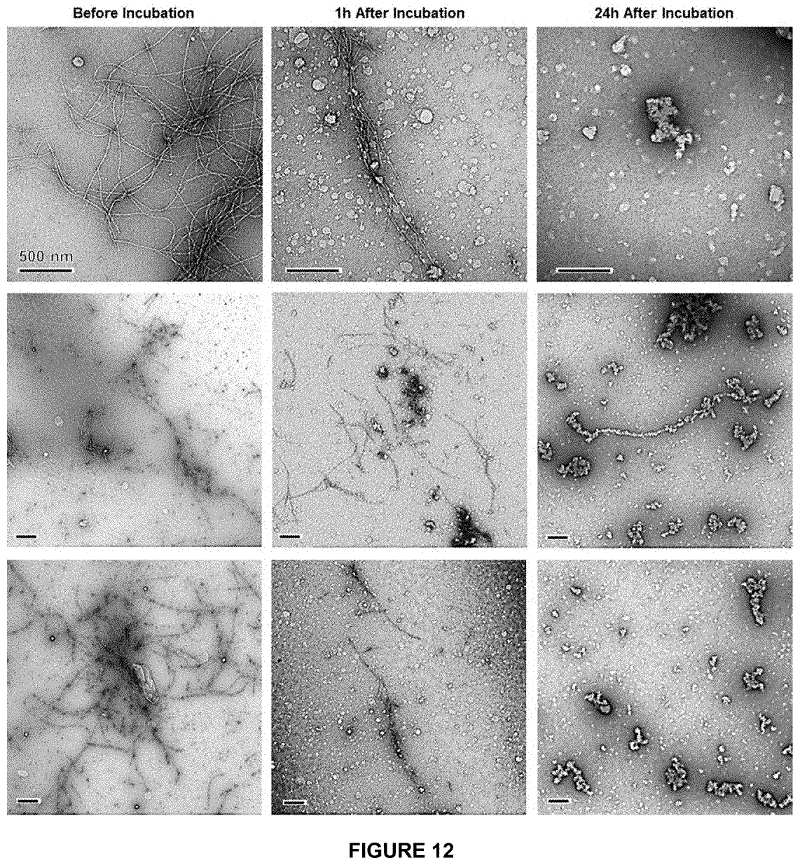

Interestingly, the vesicles were typically observed to be in close contact with fibrils (FIGS. 4B and C), suggesting the presence of explicit vesicle-fibril interactions, which are likely involved in fibril degradation. These observations support the hypothesis that fibril degradation via the chaperone-like activity of the vesicles involves the fragmentation of fibrils back into soluble protofilaments that may be further degraded into monomeric Aβ For comparison to the images with fibrils, FIG. 11 shows images of vesicles without fibrils. Finally, in addition to a decrease in mean fibril diameter, TEM images at lower magnification showed a reduction in the number of fibrils following incubation with vesicles at 1 and 24 h (FIG. 12). The reduction in the number of fibrils in the TEM images is consistent with the quantification of the relative concentration of soluble protofilaments from ThT assays.

### Example 5: Stabilizing Effect of the Vesicles to Prevent Protein Fibrillation

The present inventors next sought to understand the stabilizing effect of the lipid vesicles of the invention on proteins generally. This was investigated using insulin and α-synuclein as additional exemplary proteins and characterizing the extent to which the mixed lipid vesicles prevented their fibrillation. Notably, the aggregation of insulin is associated with injection localized amyloidosis, which impacts diabetic patients and can have severe clinical consequences. Additionally, the formation of amyloid fibers via α-synuclein is associated with Parkinson's disease and generates oligomeric species of α-synuclein that are believed to be highly toxic (similar to for amyloid-β in the case of Alzheimer's disease). As such, while insulin and α-synuclein are model proteins for studying the effect of the vesicles, they also have clinical significance. For these embodiments, a plurality of mixed lipid vesicles composition of the invention were used, including 100% DOPG, 75% DOPG/25% DOPC, 50% DOPG/50% DOPC, 25% DOPG/75% DOPC, 100% DOPC.

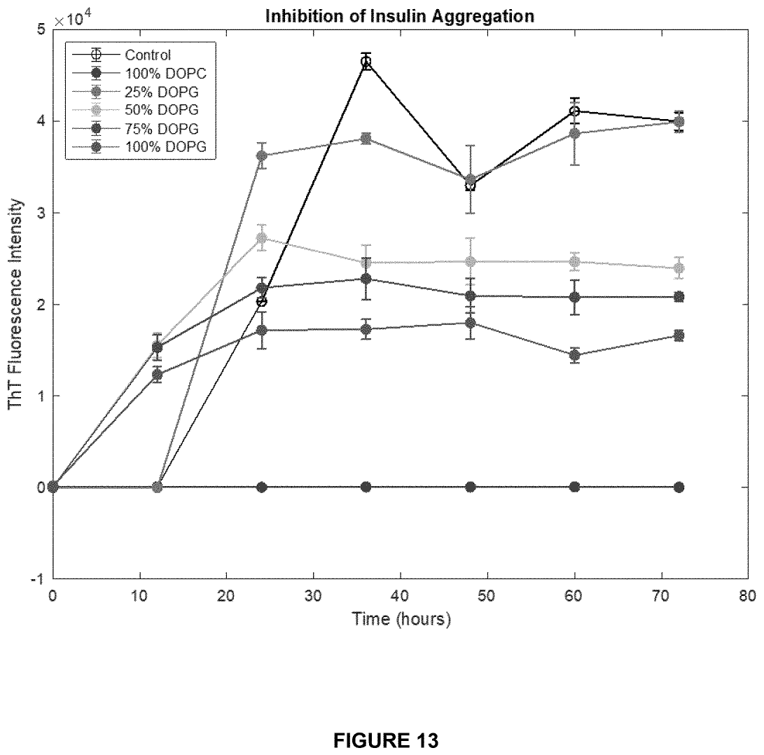

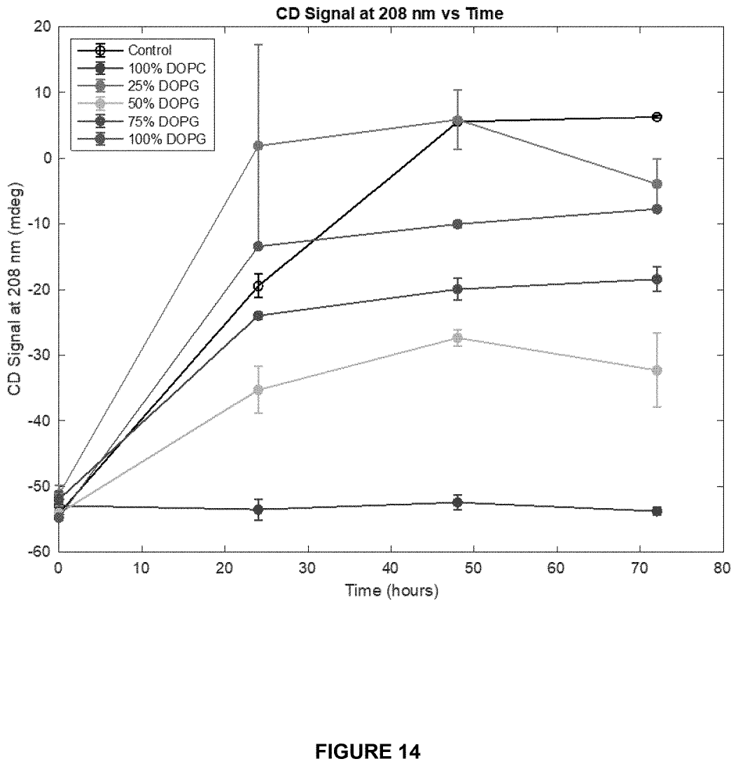

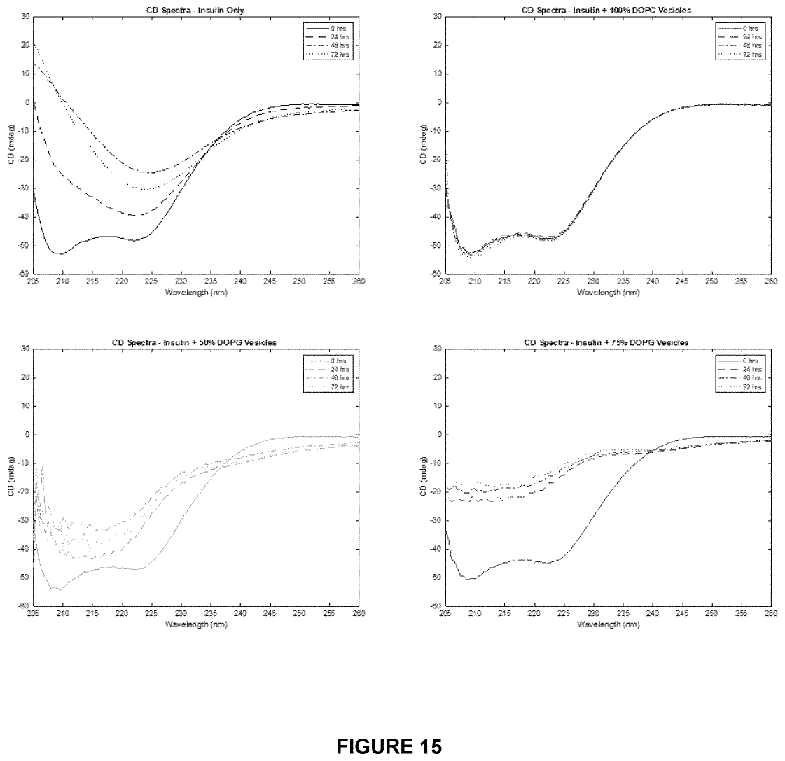

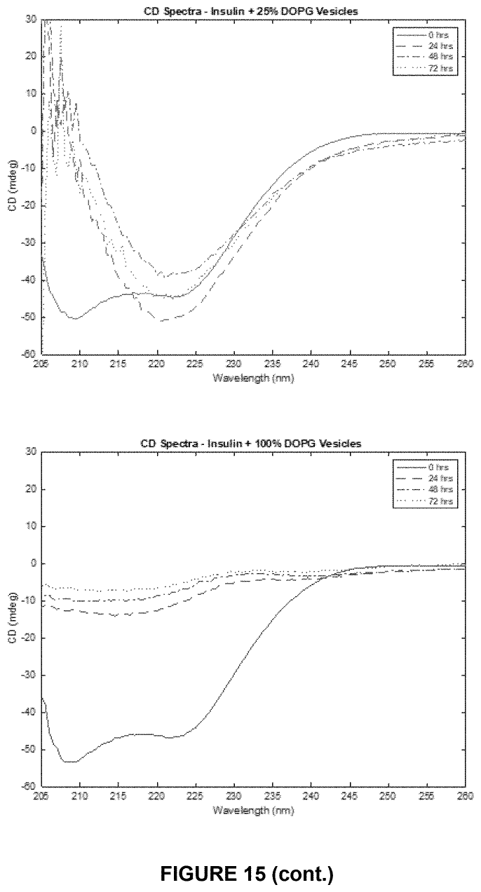

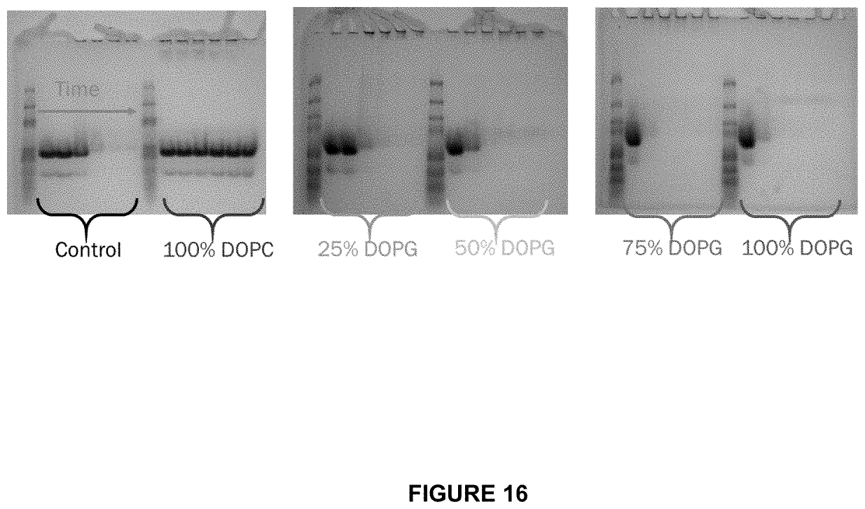

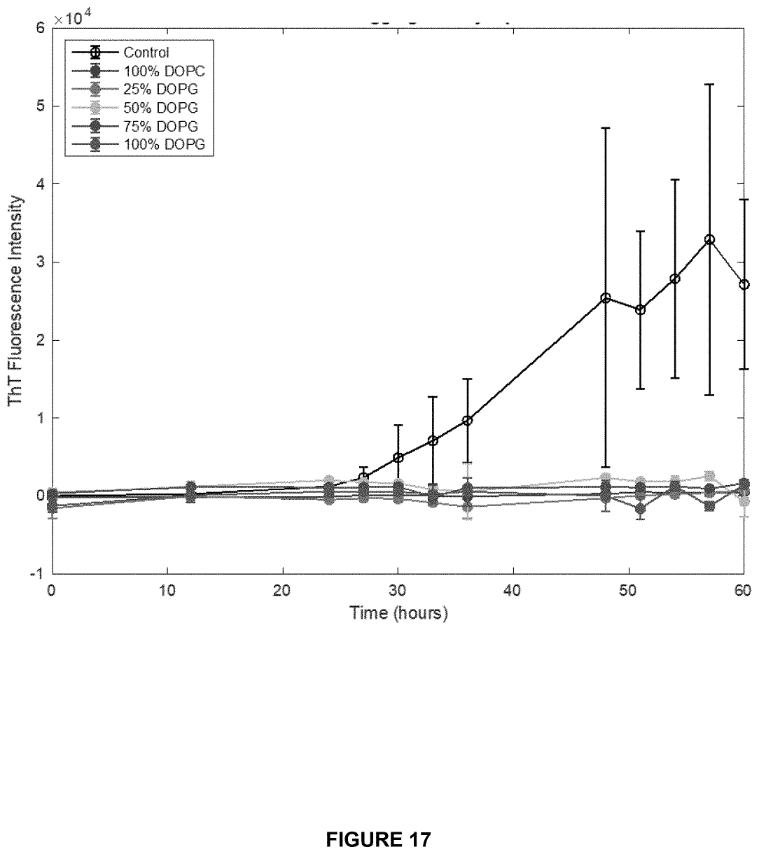

The inhibition of fibrillation of insulin and α-synuclein by the mixed vesicles was characterized using thioflavin T (ThT) as a fluorescent reporter as previously described above. As generally described in FIGS. 13 and 17, each protein was incubated with mixed vesicles at 37° C. and the change in ThT fluorescence was monitored over time. Importantly, ThT is a fluorescent dye that is non-fluorescent in solution, but fluoresces strongly upon binding to protein aggregations. As such, ThT fluorescence provides an indirect measure of protein aggregation. In addition to ThT, as shown generally in FIGS. 14-16, amyloid fiber formation for insulin was monitored using circular dichroism (CD) and native polyacrylamide gel electrophoresis (PAGE). The present inventors specifically used CD to monitor the retention of secondary structure, including α-helical content, of insulin in the presence of the vesicles. This is relevant since the loss of structure is the initial step in amyloid formation. Native PAGE provides a complementary method to observe insulin aggregation based on the difference in mobility of monomeric versus oligomeric insulin in a polyacrylamide gel.

The results of ThT analysis indicate that the vesicles also (like for amyloid-β) had a protective effect on both insulin and α-synuclein. While several of the vesicle compositions inhibited the formation of amyloid fibers by insulin, the 100% DOPC composition had a particularly dramatic stabilizing effect. This was apparent by the complete lack of amyloid fiber formation over the entire time course of the assay. Consistent with this observation, the results of CD analysis showed that insulin retained all its α-helical structure in the presence of the 100% DOPC vesicles. Notably, the retention of α-helical structure was specifically determined by monitoring the CD signal at 208 nm, which is a characteristic signal for α-helices. In the case of both ThT and CD, the results further showed that not only did several of the compositions inhibit the rate of formation of amyloid fibers by insulin, but they also appeared to reduce the maximum extent of fiber formation (as evident by the lower plateau in both the plots for ThT and CD signals). Furthermore, these observations were confirmed by native PAGE, which also showed that native insulin was retained entirely over the same time as the ThT and CD analysis (i.e., 72 hours). Whereas the band for native insulin was retained in the presence of 100% DOPC vesicles, this band disappeared over time for the control and in the presence of the other vesicles. Interestingly, for α-synuclein, all compositions of the vesicles completely inhibited amyloid fiber formation as evident by ThT analysis. This suggests that all compositions were equally as effective in protecting α-synuclein against fibrillation, and that there was not a clear optimum composition.

### Example 6: Materials and Methods

AD Peptide and Fibril Preparation: Monomeric Aβ was prepared by initially dissolving Aβ (residues 1-42) (Anaspec) in hexafluoro-2-propanol with a final protein concentration of 1 mg/mL. After dissolution, the monomeric peptide was dried under a stream of nitrogen gas and subsequently redissolved and dried a total of three times to disaggregate any fibrillar structures. Following treatment with hexafluoro-2-propanol, the dried peptide was resuspended in 20 mM Tris/HCl (pH 8.0 with 50 mM NaCl) to a final concentration of 222 μM prior to use. To induce the formation of mature Aβ fibrils, monomeric Aβ was diluted to a final concentration of 44 μM and subsequently incubated at 37° C. for 3 days while rotating gently. Following incubation, the formation of fibrils was confirmed via measuring the fluorescence of ThT as described below. Prior to use, the fibrils were serially diluted with 20 mM Tris/HCl buffer (pH 8.0 with 50 mM NaCl) with or without vesicles to the specified concentration and used immediately to prevent any further change in fibril structure.

Vesicle Preparation: Homogeneous dispersions of small unilamellar vesicles were prepared by dissolving DOPC and DOPG (Avanti Polar Lipids) separately in neat chloroform. After dissolution in chloroform, the lipids were mixed at a DOPG-to-DOPC ratio of 1:0; 3:1; 1:1, and 1:3. The organic solvent was removed via drying under a stream of nitrogen gas, followed by resuspension of the lipids in 20 mM Tris/HCl (pH 8.0 with 50 mM NaCl) to obtain vesicles with a final total phospholipid concentration of 30 mM. Finally, the solution of lipids was pulse sonicated while in an ice bath for a total of 4 min with 4 s on and 4 s off using a Misonix XL2020 probe sonicator. The resulting vesicle solutions were stored at 4° C. and used within 3 days of preparation to prevent vesicle fusion.

ThT assay: Monomeric Aβ or mature peptide fibrils (28 μM) were incubated for 0.25, 0.5, 1, 1.75, 2, 2.5, or 4 h with vesicles with a final lipid concentration of 11 mM unless otherwise specified in 20 mM Tris/HCl (pH 8.0 with 50 mM NaCl) buffer at 37° C. During incubation, the solution containing Aβ peptide/fibrils was rotated gently to ensure mixing. Following incubation, ThT (Sigma Aldrich) was added to the sample solution at a final concentration of 5 μM, which was thoroughly mixed via aspiration using a pipette. The fluorescence emission of ThT was measured using an Infinite 200 PRO (Tecan Life Sciences) microplate reader at 37° C., using an excitation and emission wavelength of 450 nm and 482 nm, respectively. Relative F_{ThT} was calculated as the fluorescence at time t divided by the fluorescence prior to incubation.

For model analysis of k_{g}′ and k_{d}, the relative ThT fluorescence was fit to the pseudo-first order model as described by Equation 1:

$$\frac{{dP}_{f}}{dt} = - {k}_{d} {P}_{f} + {k}_{g}^{′} ( 1 - {P}_{f} ) \qquad ( 1 )$$

In this model, P_{f} represents the fraction of Aβ peptide in fibrillar form, and 1−P_{f} represents the fraction of Aβ peptide in monomeric form. The integrated model, with initial boundary condition P_{f}(0)=1, is:

$${P}_{f} = \frac{{k}_{d}}{{k}_{d} + {k}_{g}^{′}} exp ( - t ( {k}_{d} + {k}_{g}^{′} ) ) + \frac{{k}_{g}}{{k}_{d} + {k}_{g}^{′}} \qquad ( 2 )$$

In the integrated form of the model (Equation 2), P_{f} corresponds to relative F_{ThT} with k_{g}′ and k_{d} representing the fitting parameters.

CD Analysis: After incubation of either monomeric or fibril Aβ (28 μM) with vesicles (11 mM lipid concentration) at 37° C. while rotating gently, CD spectra were collected from 210 to 260 nm in a 1 mm path-length quartz cell (Hellma Analytics) using a Chriscan-plus spectrometer (Applied Photophysics). Spectra of the buffer with or without vesicles was collected for each condition and used for background subtraction. Measurements were collected every 1 nm with a 0.5 s/step and a 1 nm bandwidth. For analysis, spectra were converted to mean residue ellipticity (deg cm² dmol⁻¹) and relative 0215 was determined using Equation 3:

$$Relative {θ}_{215} = \frac{{θ}_{0} - {θ}_{t}}{{θ}_{0}} \qquad ( 3 )$$

In this equation, θₜ and θ₀ represent the ellipticity of Aβ at 215 nm at time t and 0 h, respectively.

TEM Imaging: Mixtures of pre-formed Aβ fibrils (28 μM) and vesicles (11 mM lipid concentration) were imaged using a FEI T12 Spirit (Tecnai) operating at 100 kV. Aβ fibrils were formed by incubation at 37° C. under gentle rotation rotating. For sample preparation, 6 μL of the Aβ-vesicle solution was applied to a glow-discharged, carbon-coated TEM grid (copper, 200 mesh), and excess liquid was removed with filter paper. The grid was then negatively stained with 2% (w/v) uranyl acetate and allowed to dry in the open air. The width of fibers in the resulting images was determined by measuring the distance between the regions of minimum intensity on either side of the fibril. The fibril diameter was then calculated by averaging the width of the fibril at multiple locations (every 10 nm) along the fibril.

### REFERENCES

All publications and patent applications cited in the specification are herein incorporated by reference in their entirety.
(1) Knowles, T. P. J.; Vendruscolo, M.; Dobson, C. M. The Amyloid State and Its Association with Protein Misfolding Diseases. Nature Reviews Molecular Cell Biology. 2014, 15 (6), 384-396.
(2) Hartl, F. U. Protein Misfolding Diseases. Annu. Rev. Biochem. 2017, 86 (1), 21-26.
(3) Bucciantini, M.; Giannoni, E.; Chiti, F.; Baroni, F.; Taddei, N.; Ramponi, G.; Dobson, C. M.; Stefani, M. Inherent Toxicity of Aggregates Implies a Common Mechanism for Protein Misfolding Diseases. Nature. 2002, 416 (6880), 507-511.
(4) Murphy, R. M. Peptide Aggregation in Neurodegenerative Disease. Annu. Rev. Biomed. Eng. 2002, 4 (1), 155-174.
(5) Reddy, P. H. Amyloid Beta, Mitochondrial Structural and Functional Dynamics in Alzheimer's Disease. Experimental Neurology. 2009, 218 (2), 286-292.
(6) Spires-Jones, T. L.; Hyman, B. T. The Intersection of Amyloid Beta and Tau at Synapses in Alzheimer's Disease. Neuron. 2014, 82 (4), 756-771.
(7) Yip, C. M.; McLaurin, J. Amyloid-β Peptide Assembly: A Critical Step in Fibrillogenesis and Membrane Disruption. Biophys. J. 2001, 80 (3), 1359-1371.
(8) Sabaté, R.; Gallardo, M.; Estelrich, J. Spontaneous Incorporation of β-Amyloid Peptide into Neutral Liposomes. Colloids Surfaces A Physicochem. Eng. Asp. 2005, 270, 13-17.
(9) Murphy, R. M. Kinetics of Amyloid Formation and Membrane Interaction with Amyloidogenic Proteins. Biochimica et Biophysica Acta—Biomembranes. 2007, 1768 (8), 1923-1934.
(10) Yip, C. M.; Darabie, A. A.; McLaurin, J. A. Aβ42-Peptide Assembly on Lipid Bilayers. J. Mol. Biol. 2002, 318 (1), 97-107.
(11) Sani, M. A.; Gehman, J. D.; Separovic, F. Lipid Matrix Plays a Role in Abeta Fibril Kinetics and Morphology. FEBS Lett. 2011, 585 (5), 749-754.
(12) Chi, E. Y.; Ege, C.; Winans, A.; Majewski, J.; Wu, G.; Kjaer, K.; Lee, K. Y. C. Lipid Membrane Templates the Ordering and Induces the Fibrillogenesis of Alzheimer's Disease Amyloid-β Peptide. Proteins Struct. Funct. Genet. 2008, 72 (1), 1-24.
(13) Lindberg, D. J.; Wesén, E.; Björkeroth, J.; Rocha, S.; Esbjorner, E. K. Lipid Membranes Catalyse the Fibril Formation of the Amyloid-β (1-42) Peptide through Lipid-Fibril Interactions That Reinforce Secondary Pathways. Biochim. Biophys. Acta—Biomembr. 2017, 1859 (10), 1921-1929.
(14) Hellstrand, E.; Sparr, E.; Linse, S. Retardation of Aβ Fibril Formation by Phospholipid Vesicles Depends on Membrane Phase Behavior. Biophys. J. 2010, 98 (10), 2206-2214.
(15) Kinoshita, M.; Kakimoto, E.; Terakawa, M. S.; Lin, Y.; Ikenoue, T.; So, M.; Sugiki, T.; Ramamoorthy, A.; Goto, Y.; Lee, Y. H. Model Membrane Size-Dependent Amyloidogenesis of Alzheimer's Amyloid-β Peptides. Phys. Chem. Chem. Phys. 2017, 19 (24), 16257-16266.
(16) Sabaté, R.; Espargaró, A.; Barbosa-Barros, L.; Ventura, S.; Estelrich, J. Effect of the Surface Charge of Artificial Model Membranes on the Aggregation of Amyloid β-Peptide. Biochimie. 2012, 94 (8), 1730-1738.
(17) Ji, S. R.; Wu, Y.; Sui, S. fang. Cholesterol Is an Important Factor Affecting the Membrane Insertion of β-Amyloid Peptide (Aβ1-40), Which May Potentially Inhibit the Fibril Formation. J. Biol. Chem. 2002, 277 (8), 6273-6279.
(18) Terzi, E.; Hölzemann, G.; Seelig, J. Interaction of Alzheimer β-Amyloid Peptide(1-40) with Lipid Membranes. Biochemistry 1997, 36 (48), 14845-14852.
(19) Chaparro Sosa, A. F.; Black, K. J.; Kienle, D. F.; Kaar, J. L.; Schwartz, D. K. Engineering the Composition of Heterogeneous Lipid Bilayers to Stabilize Tethered Enzymes. Adv. Mater. Interfaces 2020. (In Press) https://doi.org/10.1002/admi.202000533.
(20) Schmidt, M.; Sachse, C.; Richter, W.; Xu, C.; Fandrich, M.; Grigorieff, N. Comparison of Alzheimer Aβ(1-40) and Aβ(1-42) Amyloid Fibrils Reveals Similar Protofilament Structures. Proc. Natl. Acad. Sci. 2009, 106 (47), 19813-19818.
(21) Naslund, J.; Schierhorn, A.; Hellman, U.; Lannfelt, L.; Roses, A. D.; Tjernberg, L. O; Silberring, J.; Gandy, S. E.; Winblad, B.; Greengard, P.; et al. Relative Abundance of Alzheimer A Beta Amyloid Peptide Variants in Alzheimer Disease and Normal Aging. Proc. Natl. Acad. Sci. 1994, 91 (18), 8378-8382.
(22) Klein, A. M.; Kowall, N. W.; Ferrante, R. J. Neurotoxicity and Oxidative Damage of Beta Amyloid 1-42 versus Beta Amyloid 1-40 in the Mouse Cerebral Cortex. Ann. N.Y. Acad. Sci. 1999, 893, 314-320.
(23) Chaparro Sosa, A. F.; Kienle, D. F.; Falatach, R. M.; Flanagan, J.; Kaar, J. L.; Schwartz, D. K. Stabilization of Immobilized Enzymes via the Chaperone-Like Activity of Mixed Lipid Bilayers. ACS Appl. Mater. Interfaces 2018, 10 (23), 19504-19513.
(24) Serpell, L. C. Alzheimer's Amyloid Fibrils: Structure and Assembly. Biochimica et Biophysica Acta—Molecular Basis of Disease. 2000, 1502 (1), 16-30.
(25) Tycko, R. Molecular Structure of Amyloid Fibrils: Insights from Solid-State NMR. Quarterly Reviews of Biophysics. 2006, 39 (1), 1-55.
(26) Petkova, A. T.; Yau, W. M.; Tycko, R. Experimental Constraints on Quaternary Structure in Alzheimer's β-Amyloid Fibrils. Biochemistry. 2006, 45 (2), 498-512.
(27) Lührs, T.; Ritter, C.; Adrian, M.; Riek-Loher, D.; Bohrmann, B.; Döbeli, H.; Schubert, D.; Riek, R. 3D Structure of Alzheimer's Amyloid-β(1-42) Fibrils. Proc. Natl. Acad. Sci. 2005, 102 (48), 17342-17347.
(28) Fitzpatrick, A. W. P.; Debelouchina, G. T.; Bayro, M. J.; Clare, D. K.; Caporini, M. A.; Bajaj, V. S.; Jaroniec, C. P.; Wang, L.; Ladizhansky, V.; Müller, S. A.; et al. Atomic Structure and Hierarchical Assembly of a Cross-β Amyloid Fibril. Proc. Natl. Acad. Sci. 2013, 110 (14), 5468-5473.
(29) Martins, I. C.; Kuperstein, I.; Wilkinson, H.; Maes, E.; Vanbrabant, M.; Jonckheere, W.; Van Gelder, P.; Hartmann, D.; D'Hooge, R.; De Strooper, B.; et al. Lipids Revert Inert Aβ Amyloid Fibrils to Neurotoxic Protofibrils That Affect Learning in Mice. EMBO J. 2008, 27 (1), 224-233.
(30) Shea, D.; Hsu, C. C.; Bi, T. M.; Paranjapye, N.; Childers, M. C.; Cochran, J.; Tomberlin, C. P.; Wang, L.; Paris, D.; Zonderman, J.; et al. α-Sheet Secondary Structure in Amyloid β-Peptide Drives Aggregation and Toxicity in Alzheimer's Disease. Proc. Natl. Acad. Sci. U.S.A 2019, 116 (18), 8895-8900.
(31) Friedman, R.; Pellarin, R.; Caflisch, A. Soluble Protofibrils as Metastable Intermediates in Simulations of Amyloid Fibril Degradation Induced by Lipid Vesicles. J. Phys. Chem. Lett. 2010, 1, 471-474.
(32) Godyń, J.; Jończyk, J.; Panek, D.; Malawska, B. Therapeutic Strategies for Alzheimer's Disease in Clinical Trials. Pharmacol. Reports. 2016, 68 (1), 127-138.
(33) Lorenzo, A.; Yankner, B. A. Beta-Amyloid Neurotoxicity Requires Fibril Formation and Is Inhibited by Congo Red. Proc. Natl. Acad. Sci. 1994, 91 (25), 12243-12247.
(34) Schwarzman, A. L.; Gregori, L.; Vitek, M. P.; Lyubski, S.; Strittmatter, W. J.; Enghilde, J. J.; Bhasin, R.; Silverman, J.; Weisgraber, K. H.; Coyle, P. K. Transthyretin Sequesters Amyloid Beta Protein and Prevents Amyloid Formation. Proc. Natl. Acad. Sci. 1994, 91 (18), 8368-8372.
(35) Habchi, J.; Chia, S.; Limbocker, R.; Mannini, B.; Ahn, M.; Perni, M.; Hansson, O.; Arosio, P.; Kumita, J. R.; Kumar Challa, P.; et al. Systematic Development of Small Molecules to Inhibit Specific Microscopic Steps of Aβ42 Aggregation in Alzheimer's Disease. Proc. Natl. Acad. Sci. 2017. 114 (2), 200-208.
(36) Underwood, E. Alzheimer's Amyloid Theory Gets Modest Boost. Science. 2015, 349, 464-464.
(37) Doig, A. J.; Derreumaux, P. Inhibition of Protein Aggregation and Amyloid Formation by Small Molecules. Curr. Opin. Struct. Biol. 2015, 30, 50-56.
(38) Barr, R. K.; Verdile, G.; Wijaya, L. K.; Morici, M.; Taddei, K.; Gupta, V. B.; Pedrini, S.; Jin, L.; Nicolazzo, J. A.; Knock, E.; et al. Validation and Characterization of a Novel Peptide That Binds Monomeric and Aggregated β-Amyloid and Inhibits the Formation of Neurotoxic Oligomers. J. Biol. Chem. 2016, 291 (2), 547-559.
(39) Cho, P. Y.; Joshi, G.; Johnson, J. A.; Murphy, R. M. Transthyretin-Derived Peptides as β-Amyloid Inhibitors. ACS Chem. Neurosci. 2014, 5 (7), 542-551.
(40) Griner, S. L.; Seidler, P.; Bowler, J.; Murray, K. A.; Yang, T. P.; Sahay, S.; Sawaya, M. R.; Cascio, D.; Rodriguez, J. A.; Philipp, S.; et al. Structure Based Inhibitors of Amyloid Beta Core Suggest a Common Interface with Tau. Elife. 2019, 8, e46924.
(41) Bulbake, U.; Doppalapudi, S.; Kommineni, N.; Khan, W. Liposomal formulations in clinical use: an updated review. Pharmaceutics. 2017, 9(2), 12.
(42) Liu, Y.; Fang, J.; Kim, Y. J.; Wong, M. K.; Wang, P. Codelivery of doxorubicin and paclitaxel by cross-linked multilamellar liposome enables synergistic antitumor activity. Mol. Pharm. 2014, 11(5), 1651-1661.
(43) Hanson, M. C.; Abraham, W.; Crespo, M. P.; Chen, S. H.; Liu, H.; Szeto, G. L.; Kim, M.; Reinherz, E. L.; Irvine, D. J. Liposomal vaccines incorporating molecular adjuvants and intrastructural T-cell help promote the immunogenicity of HIV membrane-proximal external region peptides. Vaccine. 2015, 33(7), 861-868.

1. A composition to promote protein re-folding comprising a lipid bilayer composed of zwitterionic and/or anionic phospholipids, wherein said lipid bilayer catalytically promotes protein re-folding associated with a disease or disorder.

2. The composition of claim 1, wherein said zwitterionic phospholipid comprises 1,2-dioleoyl-sn-glycero-3-phosphocholine (DOPC).

3. The composition of claim 1, wherein said anionic phospholipid comprises 1,2-dioleoyl-sn-glycero-3-phospho-(1′-rac-glycerol) (DOPG).

4. The composition of claim 1, wherein said lipid bilayer comprises unilamellar vesicle having a lipid bilayer.

5. The composition of claim 1, wherein said lipid bilayer comprises a lipid bilayer disposed on a nanoparticle configured to secure said lipid bilayer.

6. The composition of claim 1, wherein said lipid bilayer comprises a heterogeneous lipid bilayer of DOPG and DOPC.

7. The composition of claim 6, wherein said heterogeneous lipid bilayer of DOPG and DOPC comprises a heterogeneous lipid bilayer selected from the group consisting of:
a heterogenous lipid bilayer having 99% DOPG and 1% DOPC;
a heterogenous lipid bilayer having 75% DOPG and 25% DOPC;
a heterogenous lipid bilayer having 50% DOPG and 50% DOPC;
a heterogenous lipid bilayer having 25% DOPG and 75% DOPC;
a heterogenous lipid bilayer having 1% DOPG and 99% DOPC; and
a heterogenous lipid bilayer having between 99-1% DOPG and between 1-99% DOPC.

8. The composition of claims 1-5, wherein said lipid bilayer comprises a homogenous lipid bilayer of either DOPG or DOPC.

9. The composition of claims 1-8, wherein said protein associated with a disease or disorder comprises amyloid-β (Aβ).

10-15. (canceled)

16. A composition for inhibiting the formation of protein aggregates or reversing pre-formed protein aggregates comprising a lipid bilayer composed of zwitterionic and/or anionic phospholipids, wherein said lipid bilayer catalytically inhibits or reverses the formation of said protein aggregates associated with a disease or disorder.

17. The composition of claim 16, wherein said zwitterionic phospholipid comprises 1,2-dioleoyl-sn-glycero-3-phosphocholine (DOPC).

18. The composition of claim 16, wherein said anionic phospholipid comprises 1,2-dioleoyl-sn-glycero-3-phospho-(1′-rac-glycerol) (DOPG).

19. The composition of claim 16, wherein said lipid bilayer comprises unilamellar vesicle having a lipid bilayer.

20. The composition of claim 16, wherein said lipid bilayer comprises a lipid bilayer disposed on a nanoparticle configured to secure said lipid bilayer.

21. The composition of claim 16, wherein said lipid bilayer comprises a heterogeneous lipid bilayer of DOPG and DOPC.

22. The composition of claim 21, wherein said heterogeneous lipid bilayer of DOPG and DOPC comprises a heterogeneous lipid bilayer selected from the group consisting of:
a heterogenous lipid bilayer having 99% DOPG and 1% DOPC;
a heterogenous lipid bilayer having 75% DOPG and 25% DOPC;
a heterogenous lipid bilayer having 50% DOPG and 50% DOPC;
a heterogenous lipid bilayer having 25% DOPG and 75% DOPC;
a heterogenous lipid bilayer having 1% DOPG and 99% DOPC; and
a heterogenous lipid bilayer having between 99-1% DOPG and between 1-99% DOPC.

23. The composition of claim 16, lipid bilayer comprises a homogenous lipid bilayer of either DOPG or DOPC.

24. The composition of claim 16, wherein the protein aggregates comprises amyloid-β (Aβ) fibrils.

25. (canceled)

26. The composition of claim 24, wherein the β-sheet structure of said Aβ fibril is decreased.

27-55. (canceled)

56. A composition comprising a heterogeneous lipid bilayer composed of (DOPC) and (DOPG) wherein said lipid bilayer catalytically:
promotes protein re-folding of tau, α-synuclein, and/or tumor suppressor protein p53; or
reverses the formation of said protein aggregates of tau, α-synuclein, and/or tumor suppressor protein p53; or
inhibits the formation of said protein aggregates of tau, α-synuclein, and/or tumor suppressor protein p53.

57-166. (canceled)
